(19) United States
(12) Patent Application Publication
(10) Pub. No.: US 2025/0154554 A1
(43) Pub. Date: May 15, 2025

(54) ANTIMICROBIAL SUSCEPTIBILITY

(71) Applicant: FASTinov, SA, Porto (PT)

(72) Inventor: Cidalia AZEVEDO PINA VAZ, Porto (PT)

(21) Appl. No.: 18/837,030

(22) PCT Filed: Feb. 9, 2023
(86) PCT No.: PCT/EP2023/053187

§ 371 Date: Aug. 8, 2024

(30) Foreign Application Priority Data
Feb. 10, 2022 (GB) 2201697.6

(51) Int. Cl. C12Q 1/18; C12Q 1/04; C12Q 1/24

(52) U.S. Cl. CPC C12Q 1/18; C12Q 1/04; C12Q 1/24

(57) ABSTRACT

The invention relates to methods and kits for the detection and determination of the susceptibility of microorganisms to therapeutic agents. The invention relates to the rapid determination of bacterial susceptibility to antibiotics. The invention also extends to methods of sample preparation and microorganism extraction from a biological sample, and to methods of identifying microorganisms, and to kits and panels specifically designed to determine the susceptibility of extracted microorganisms to antimicrobial agents. The invention is especially useful in clinical diagnostic and veterinary medicine.

### RELATED APPLICATIONS

The present application is a U.S. national phase application under 35 U.S.C. § 371 of International Application No. PCT/EP2023/053187, filed on Feb. 9, 2023 and published as WO 2023/152218 A1 on Aug. 17, 2023; which claims the priority of GB Application No. 2201697.6, filed on Feb. 10, 2022. The content of each of these related applications is incorporated herein by reference in its entirety.

The present invention relates to antimicrobial susceptibility, and to methods and kits for the detection and determination of the susceptibility of microorganisms to therapeutic agents and, particularly, although not exclusively, to the rapid determination of bacterial susceptibility to antibiotics. The invention also extends to methods of sample preparation and microorganism extraction from a biological sample, and to methods of identifying microorganisms, and to kits and panels specifically designed to determine the susceptibility of extracted microorganisms to antimicrobial agents. The invention is especially useful in clinical diagnostic and veterinary medicine.

Antimicrobial resistance poses a serious global threat of increasing concern to human, animal, and environment health. One of the main causes of antimicrobial resistance includes excessive use of antibiotics in animals and humans, and release of non-metabolized antibiotics or their residues into the environment through manure or faeces.

The antimicrobial susceptibility profile of a microorganism is usually determined by evaluating its growth ability in the presence of different antimicrobials, which usually takes around two days from the biological sample collection to the determination of the susceptibility profile. Therapy based on epidemiology, through application of broad-spectrum antimicrobials, is applied in most hospitals worldwide because of the long time required for these procedures. This in turn triggers antimicrobial resistance leading to severe individual and public health threats.

Therefore, there is an urgent need for rapid Antimicrobial Susceptibility Tests (AST) that are able to provide, in a timely manner, relevant and useful information that can enable the optimal implementation of antimicrobial therapy on the basis of a scientific report. To respond to this increasing need, several approaches have been developed over the years, including molecular based assays. Although molecular assays may have the advantages of screening directly on polymicrobial samples for selected antimicrobial resistance mechanisms, they have a very limited role in patient treatment, because they do not provide information regarding the susceptibility of the bacteria to certain antimicrobials.

Furthermore, molecular assays can only assess mechanisms of resistance and there has been documented evidence of discrepancies between genotypic and phenotypic tests outcomes. Antimicrobial resistance is too complex to rely on such methods for a truly comprehensive understanding of the problem.

Therefore, to overcome the limitation of the existing ASTs, the inventors have developed a new and highly innovative method termed the “FASTinov assay” together with corresponding platforms, which allow the provision of an AST report to clinicians in record time directly from a positive biological sample, and therefore, result in the implementation of a targeted antimicrobial therapy instead of the use of broad-spectrum antimicrobials [6,7,8]. The present invention also enables the detection of a multi-resistant microorganism [5], which in turn allows the implementation of a more aggressive therapy in the least amount of time, thereby avoiding its spreading through a hospital ward, the whole hospital or beyond. Accordingly, the invention described herein has the potential to revolutionise treatment of infected patients with significant advantages for both patients and society.

The present invention presents a major improvement and development of the inventors' prior susceptibility testing methods and panels described in WO2012/164547 A1 [1]. The inventors' new method involves an innovative sample preparation and extraction process aiming to separate microorganisms from human or animal cells and debris from a positive biological sample. The isolated microorganism is then incubated with antimicrobial drugs, for a very short period of time, before being so stained with optimized fluorescent probes. The lesions produced by the drugs on the cells are then evaluated using a flow cytometer through a multiparametric analysis. Because the test is growth-independent, the results are obtained in record time.

Flow cytometry has been proven to be a powerful tool in a variety of disciplines, such as haematology and cytopathology, and its increasing use in microbiology has significant potential. Indeed, using flow cytometry, cell microbial populations can be discriminated in terms of susceptibility versus resistant phenotypes in a very short time-frame. In fact, using the correct fluorochromes at determined wavelengths, antimicrobial effects can be quickly detected and quantified using software that allows for the analysis of large amount of biological data, including cell size and complexity.

Accordingly, in a first aspect of the invention, there is provided a method for determining the susceptibility phenotype, to at least one therapeutic agent, of a microorganism present in a biological sample, the method comprising:
(i) introducing a biological sample comprising a microorganism into each of:
   (a) one or more of a first, test reservoir,
   (b) one or more of a second, positive control reservoir, and
   (c) one or more of a third, negative control reservoir, wherein the negative control reservoir comprises non-viable microorganisms;
(ii) contacting the biological sample in the one or more first, test reservoir with at least one therapeutic agent;
(iii) contacting the biological sample in the one or more first, second and third reservoir with at least one fluorescent marker; and
(iv) performing a fluorescence analysis in order to obtain one or more fluorescence parameters for the biological sample in each of the reservoirs,
   wherein the microorganism's susceptibility phenotype to the at least one therapeutic agent is obtained by comparing one or more fluorescence parameters between the reservoirs.

Advantageously, the inventors have discovered that the use of the third, negative control reservoir used in addition to the second, positive control reservoir in the method significantly increases the sensitivity of the assay, resulting in effective and accurate susceptibility phenotype readouts, enabling optimum therapeutic treatment for patients. This is because the use of both positive and negative controls, in addition to the test sample, ensures that only viable microorganisms are considered in the susceptibility phenotype assessment assay. Non-viable (i.e. dead) microorganisms cannot respond to the therapeutic agent, and so, if they were included in the susceptibility phenotype assessment, such a non-viable microorganism could wrongly appear resistant to all therapeutic agents, which would correspond to a false negative or major error, and consequently result in the implementation of the wrong subsequent therapy.

Preferably, therefore, the one or more third, negative control reservoir comprises non-viable or dead microorganisms, and the one or more second, positive control reservoir comprises viable or living microorganisms. Preferably, the one or more first, test reservoir comprises viable or living microorganisms.

Preferably, the one or more first test reservoir comprises at least 60%, 70%, or 80% viable cells. More preferably, the one or more first test reservoir comprises at least 85%, 90%, or 95% viable cells. More preferably, the one or more first test reservoir comprises at least 96%, 97%, 98%, 99% or 100% viable cells. Preferably, the one or more first test reservoir comprises at least 10⁵ CFU/ml of viable cells, or at least 10⁶ CFU/ml of viable cells, or at least 10⁷ CFU/ml of viable cells.

Preferably, the one or more second positive control reservoir comprises at least 60%, 70%, or 80% viable cells. More preferably, the one or more first second positive control reservoir comprises at least 85%, 90%, or 95% viable cells. More preferably, the one or more second positive control reservoir comprises at least 96%, 97%, 98%, 99% or 100% viable cells. Preferably, the one or more second test reservoir comprises at least 10⁵ CFU/ml of viable cells, or at least 10⁶ CFU/ml of viable cells, or at least 10⁷ CFU/ml of viable cells.

Preferably, the one or more third negative control reservoir comprises at least 60%, 70%, or 80% non-viable cells. More preferably, the one or more third negative control reservoir comprises at least 85%, 90%, or 95% non-viable cells. More preferably, the one or more third negative control reservoir comprises at least 96%, 97%, 98%, 99% or 100% non-viable cells.

The cells in the one or more third negative control may be rendered non-viable by exposure to a cell-killing agent. Preferably, the one or more third negative control reservoir comprises the cell-killing agent. Preferably, the cell-killing agent is selected from a group consisting of: ethanol, 2-phenoxyethanol, citric acid, and benzydamine hydrochloride. Most preferably, the cell-killing agent is benzydamine hydrochloride.

The inventors had previously shown that benzydamine hydrochloride rapidly kills bacteria [14] and fungi [13] by cell membrane lesions. The objective of this control is to ensure that the fluorescent probe is active and has a maximum effect when the cells are killed by benzydamine hydrochloride. Therefore, in a preferred embodiment, the cells in the one or more third negative control reservoir are made non-viable by exposure to benzydamine hydrochloride.

If the at least one fluorescent marker is not present or is not performing well, the effect of the therapeutic agent on the microorganism will be difficult, if not impossible, to assess. Therefore, the use of the negative control reservoir enhances the accuracy of the method. Furthermore, the inventors have surprisingly discovered that the use of both positive and negative controls in the methods of the invention enables the identification of different levels of susceptibility phenotype to the at least one therapeutic agent, so namely susceptible, intermediate or resistant to the at least one therapeutic agent.

Accordingly, in one embodiment, the susceptibility phenotype may be susceptible, intermediate or resistant.

The fluorescence analysis may be performed by flow cytometry. The accurate measurement of fluorescent signal by flow cytometry can be hampered by background fluorescence caused by the microorganism's own auto-fluorescence. To overcome this problem, the inventors have advantageously discovered that the use of a fourth auto-fluorescence control reservoir comprising a microorganism untreated with the therapeutic agent and unstained with the fluorescent marker, enables the assessment of microorganism's own auto-fluorescence. When taken into account during the analysis of the fluorescent parameters used to determine the susceptibility profile of the microorganism, removing the auto-fluorescence background significantly increases the test accuracy.

Therefore, in one embodiment, the method further comprises introducing the biological sample in one or more of a fourth auto-fluorescence control reservoir, wherein neither the therapeutic agent nor the fluorescent marker is added to the reservoir. In other words, the one or more fourth auto-fluorescence control reservoir does not comprise the therapeutic agent or the fluorescent marker.

Preferably, the one or more fourth auto-fluorescence control reservoir comprises viable or living microorganisms. Preferably, the one or more fourth auto-fluorescence control reservoir comprises at least 60%, 70%, or 80% viable cells. More preferably, the one or more fourth auto-fluorescence control reservoir comprises at least 85%, 90%, or 95% viable cells. More preferably, the one or more fourth auto-fluorescence control reservoir comprises at least 96%, 97%, 98%, 99% or 100% viable cells. Preferably, the one or more fourth auto-fluorescence control reservoir comprises at least 10⁵ CFU/ml of viable cells, or at least 10⁶ CFU/ml of viable cells, or at least 10⁷ CFU/ml of viable cells.

The inventors have surprisingly discovered that the method's sensitivity increases when the microorganism is isolated or separated from any host cells (i.e. emanating from the subject/patient from whom the sample is taken) present in the biological sample prior to its introduction into the test and control reservoirs. Without a highly pure and concentrated sample of microorganism cells (absent of any host cells from the test subject), an accurate flow cytometry reading and analysis would be difficult since the hosts' cells could themselves generate non-specific fluorescence, thereby interfering with the fluorescent analysis generated by the microorganisms.

Accordingly, in a preferred embodiment, the method further comprises a sample preparation step. Preferably, the sample preparation step comprises extracting and purifying the microorganism from host cells and/or debris also present in the biological sample before its introduction into the one or more first, second, the third, and/or optionally the fourth reservoirs.

The inventors have successfully demonstrated for the first time that a density gradient solution (e.g. Histopaque®, available from Sigma) that can separate the microorganism from the host's cells and debris through a centrifugation gradient, was much more effective than the standard physical separation used in the prior art. This novel method works with all types of biological samples without affecting the physical and physiological functions of the microorganism, such that the fluorescence analysis is unaffected.

Other methods of direct identification of microorganisms from a biological sample have been described in the art. However, these methods are limited to the use of haemolytic agents, and the use of a density gradient solution, such as Histopaque®, has never been described in clinical microbiology methodologies before for the purpose of extracting and purifying a microorganism (such as a bacterium) from a biological sample. The novel method developed by the inventors allows the purified microorganism to accumulate at the bottom of a tube or vial, while the cell debris and host's cells combined with histopaque (RTM) accumulate at the top of the tube. Furthermore, the novel method developed by the inventors enables the use the same sample for MALDI-TOF identification of bacteria causing the infection. Generally, prepared samples must undergo additional treatment method before being processing for MALDI-TOF bacterial identification. With the inventor's novel sample preparation method, however, the sample is automatically ready for MALDI-TOF identification without requiring any further treatment, thereby saving time, which is critical in clinical microbiology laboratory settings where timely diagnosis have a huge impact on the patient's prognosis.

Accordingly, the sample preparation step preferably comprises purification of the microorganism from the biological sample before it is introduced into each of the first, second, third and/or optionally the fourth reservoir. The sample preparation step may comprise:
(i) obtaining the biological sample comprising the microorganism; and
(ii) contacting the biological sample with a density gradient solution to thereby purify the microorganism.

Preferably, the density gradient solution is Histopaque®. It is preferably used pure, without any dilution. Histopaque (RTM) is a density gradient cell separation medium comprising Ficoll and sodium diatrizoate. Most preferably, the biological sample and the density gradient are present at a ratio of about 1:1. The sample preparation step may comprise extracting the microorganism from the biological sample.

After the step contacting the biological sample with a density gradient solution, the sample preparation step then preferably comprises centrifuging the sample, preferably for at least one minute, preferably at about 13,000 rpm. The sample preparation step comprises re-suspending the resulting in pellet (comprising purified microorganisms) in media. The media may preferably be cation-adjusted broth, optionally about 1 ml of sterile and filtered Mueller Hinton II cation-adjusted broth.

In some embodiments, the sample preparation step may comprise contacting the biological sample with a haemolytic agent before contacting the same with the density gradient solution. Preferably, the haemolytic agent is configured to lyse any contaminating cells in the sample, such as contaminating host cells emanating from the host from whom the sample is taken, or debris present in environmental samples.

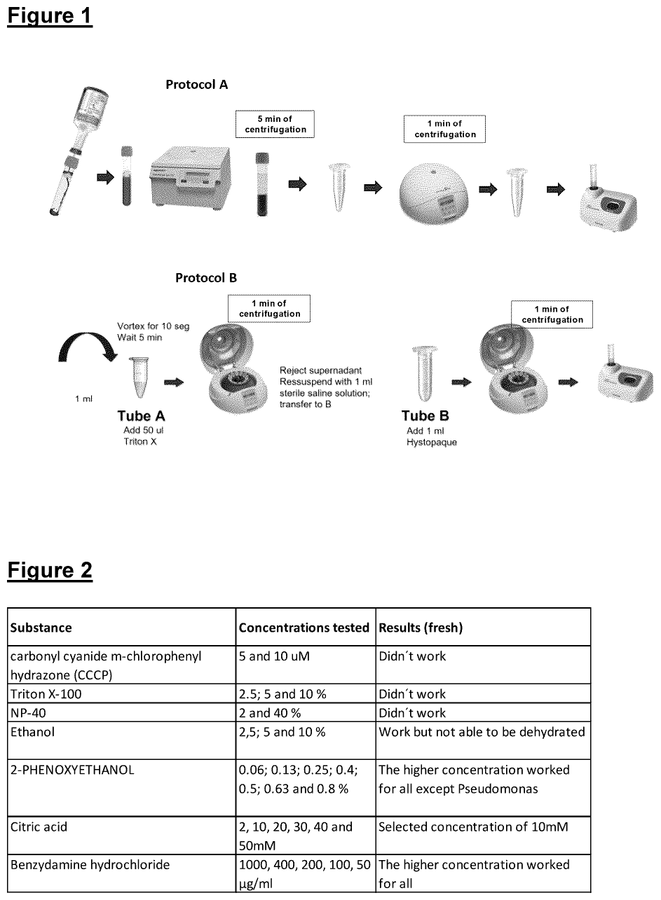

The inventors have found that the use of a haemolytic agent is particularly advantageous in embodiments in which the biological sample may comprise blood, preferably a blood culture. Protocol B disclosed in FIG. 1 illustrates one preferred embodiment of the sample preparation step of the method, when the biological sample is a blood sample.

In one embodiment, the haemolytic agent may be Triton™ X-100 (commonly called Triton X-100). Triton™ X-100 is a common non-ionic surfactant and emulsifier which is often used in biochemical applications to solubilize proteins. It is considered a comparatively mild and non-denaturing detergent. It is utilised for lysing cells to extract protein and cellular organelles. It can also permeabilise a living cell membrane for transfection, or used for DNA extraction.

Preferably, Triton X-100 is used at a concentration of between 0.1% and 3% (v/v), or between 0.2% and 2.5% (v/v) or between 0.3% and 2% (v/v), or between 0.4 and 1% (v/v), and preferably about 0.5% (v/v). Preferably, the biological sample is contacted with the haemolytic agent (preferably Triton X-100) for at least 1 minute, 2 minutes or 5 minutes. Preferably, the biological sample is contacted with the haemolytic agent (preferably Triton X-100) for less than 2 hours, 1 hour, 30 minutes or less than 15 minutes. This step may be carried out at room temperature. Thus, preferably the sample preparation step comprises contacting the biological sample with a haemolytic agent for five minutes at room temperature.

In another and preferred embodiment, the haemolytic agent is Tergitol™ 15-S-9 (commonly called Tergitol). Tergitol is a secondary alcohol ethoxylate, and a linear non-ionic surfactant.

Preferably, Tergitol is used at a concentration of between 2.5-25% (v/v), or between 3% and 22% (v/v), or between 4% and 20% (v/v), or between 5 and 18% (v/v). More preferably, Tergitol is used at a concentration of, or between 6% and 16% (v/v), or between 7% and 14% (v/v). Most preferably, Tergitol is used at a concentration of between 8 and 12% (v/v), or between 9 and 11% (v/v), and preferably about 10% (v/v). Preferably, the biological sample is contacted with Tergitol, and mixed (preferably by vortex).

Advantageously, Tergitol has a better safety profile, as it is not listed in the REACH (Registration, Evaluation, Authorisation and Restriction of Chemicals) list of dangerous substances. Furthermore, Tergitol only requires a very short incubation time, and therefore, ensures a time-efficient diagnosis. In addition, Tergitol works on MALDI-Tof identification which is advantageous. Tergitol also ensures a higher degree of purity of the sample to be analysed. Therefore, the use of Tergitol is compatible with Flow Cytometry (FC) analysis, which require extremely high purity of the cell suspension.

After contacting the biological sample with a haemolytic agent and before contacting the same with the density gradient solution and before step, the sample preparation step preferably comprises centrifuging the sample, preferably for at least one minute, preferably at about 13,000 rpm. Preferably, the sample preparation step then comprises re-suspending the resulting pellet (with the microorganisms). Preferably, the pellet is re-suspended in saline solution, optionally about 0.05 ml to 2 ml, 0.01 ml to 1 ml, or 0.25 ml to 0.75 ml of sterile and filtered saline solution. Most preferably, the pellet is re-suspended in 0.5 ml of sterile and filtered saline solution.

The haemolytic agent used in the extraction method allows the lysing of the host's cells, in particular red blood cells, and potential debris that may present in the biological sample, while preserving the microorganism's cells. The sample preparation protocol described herein allows the purified microorganisms to accumulate towards the bottom of the tube, while the cell debris and host's cells combined with density gradient solution accumulate towards the top of the tube.

The sample preparation step may further comprise identifying the microorganism before introducing the biological sample comprising the microorganism into each of the first, second, third and/or optionally the fourth reservoir. The identification of the microorganism may be performed using genetic methods, microarrays, physical methods and/or mass spectrometry methods. Genetics methods may be a quantitative Polymerase Chain Reaction (PCR), an immuno-PCR, or a combination thereof. Microarrays may be DNA microarrays, protein microarrays, antibody microarrays, or a combination thereof. Physical methods may be an infrared and Raman spectroscopy or a laser-induced breakdown spectroscopy (LIBS). Mass spectrometry methods may be performed through an ICP mass spectrometer, DART mass spectrometer, or a MALDI-TOF Brucker.

Preferably, the identification of the microorganism is performed using a mass spectrometry method. Most preferably, the mass spectrometry method is performed using a MALDI-TOF, for example from manufacturers, such as Brucker.

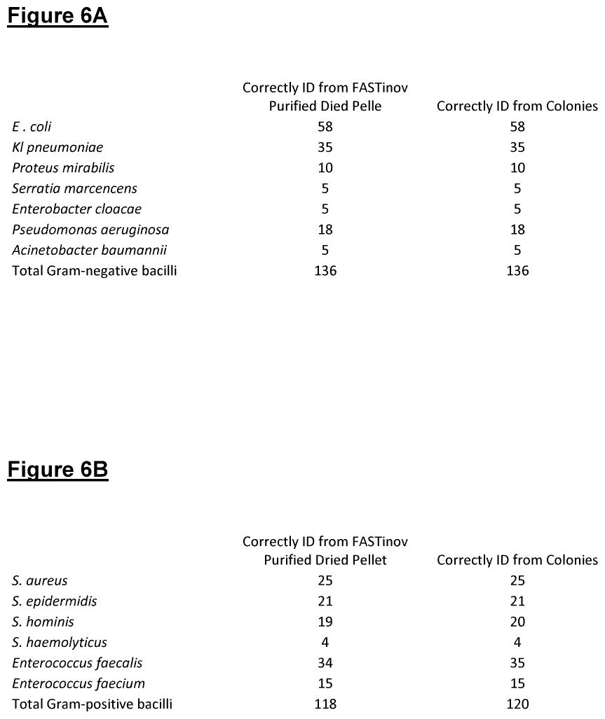

Advantageously, as shown in FIGS. 6A and 6B, the inventors have discovered that using samples extracted and purified using the above-described sample preparation step for the subsequent identification of the microorganism quickly yields (i.e. within only a few hours) outstanding results and excellent identification accuracy on a par with those obtained from standard microorganism colonies, without the extra step of generating colonies from an overnight culture.

Therefore, in one embodiment, the identification of the microorganism is performed using a sample which has been purified using the sample preparation step described above. Preferably, the purified sample is substantially dried before being exposed to a mass spectrometer.

Accordingly, the sample preparation step may further comprise an additional centrifugation cycle in order to dry the pellet. The additional centrifugation cycle may be run between 5 seconds and 5 minutes, between 10 seconds and 5 minutes, between 30 seconds and 5 minutes, between 30 seconds and 4 minutes, between 30 seconds and 3 minutes, between 30 seconds and 2 minutes, between 30 seconds and 1.5 minutes, or between 30 seconds and 1 minute. Preferably, the additional centrifugation cycle may be run for at least 1 minute.

The additional centrifugation step may be run between 5000 rpm and 20000 rpm, between 10000 rpm and 18000 rpm, between 12000 rpm and 16000 rpm, or 13000 rpm and 15000 rpm.

The sample preparation step may further comprise drying the pellet resulting from the additional centrifugation cycle. The resulting pellet may be dried between 0° C. and 40° C., or between 30° C. and 40° C., or between 35° C. and 40° C. The resulting pellet may be dried at 4° C., room temperature or at 37° C.

Other sample purification methods are also considered for the identification of the microorganism within the scope of the invention for the identification of the microorganism. These include, for example, the methods recommended by mass spectrometer manufacturers.

The inventors have successfully demonstrated that the novel sample preparation step described above advantageously provides a very fast, one-step way to obtain a highly purified sample for identifying the microorganism efficiently and then subsequently testing its susceptibility to antimicrobials in a time-efficient manner.

The biological sample may be of a human, animal or environmental origin. In embodiments in which the biological sample is of a human or animal origin, the sample is preferably a biological bodily sample taken from the test subject. The method for determining the susceptibility phenotype of the microorganism in the sample is, therefore, preferably carried out in vitro or ex vivo. The sample may comprise tissue, blood, plasma, serum, spinal fluid, urine, bronchial secretion, cerebrospinal fluid, sweat, saliva, sputum, tears, breast aspirate, prostate fluid, seminal fluid, vaginal fluid, stool, cervical scraping, amniotic fluid, intraocular fluid, mucous, moisture in breath, animal tissue, cell lysates, tumour tissue, hair, skin, buccal scrapings, nails, bone marrow, cartilage, prions, bone powder, ear wax, or combinations thereof. The sample may be a biopsy. Preferably, the sample is blood. Preferably, the sample is saliva

In a preferred embodiment, the biological sample of human origin is a urine sample or blood sample. A blood sample is preferred. Most preferably, the sample is a blood culture.

In embodiments in which the biological sample is of environmental origin, the sample is preferably selected from the group consisting of: soil; water; and plant residue.

The biological sample may be cultured or uncultured before it is subjected to the analysis of the method of the invention. Advantageously, the ability to process the so biological sample when uncultured significantly reduces the experimental time and prevents unnecessary sample culturing steps. Therefore, preferably, the biological sample is uncultured.

The biological material may be an aerobic or anaerobic sample. The sample may therefore comprise an aerobic blood culture or an anaerobic blood culture.

Prior to the fluorescent analysis, the one or more first, second, the third, and/or optionally the fourth reservoirs may be incubated between 0° C.-40° C., 20° C.-40° C., 22° C.-40° C., 24° C.-40° C., 26° C.-40° C., 28° C.-40° C., 30° C.-40° C., 32° C.-40° C., 34° C.-40° C. 36° C.-40° C., or 38° C.-40° C. The one or more first, second, the third, and/or the fourth reservoirs may be incubated between 0° C. and 40° C., or between 30° C. and 40° C., or between 35° C. and 40° C., or at around 0° C., 4° C., at room temperature, or at 37° C. Preferably, the one or more first, second, the third, and/or the fourth reservoir is incubated at 37° C.

Prior to the fluorescent analysis, the one or more first, second, the third, and/or optionally the fourth reservoirs may be incubated with shaking or without shaking. Preferably, the biological sample is incubated with shaking.

Prior to the fluorescent analysis, the one or more first, second, the third, and/or optionally the fourth reservoirs may be incubated for up to 6 hours, 5 hours, 4 hours, 3 hours, 2 hours, 1 hour or 30 minutes. Preferably, the biological sample is incubated for about 1 hour.

Following this incubation step, the one or more first, second, the third, and/or the fourth reservoirs are then preferably subjected to the fluorescent analysis step.

The microorganism may be a bacterium, a virus, a fungus or a protozoan.

In a preferred embodiment, the microorganism is a bacterium. The bacterium may be a gram-negative bacterium, a gram-positive bacterium, or a bacterium presenting the characteristics of both gram-negative and gram-positive bacteria. The bacterium may be aerobic or anaerobic. In a preferred embodiment, the microorganism is an aerobic bacterium.

Preferably, the gram-negative bacterium may be a gram-negative bacillus selected from a group consisting of: Escherichia coli ATCC 25922; Escherichia coli ATCC 8739; Escherichia coli ATCC 35218; Escherichia coli BAA 2425; Klebsiella pneumoniae ATCC 13443; Klebsiella pneumoniae BAA 1705; Klebsiella pneumoniae ATCC 700603; Klebsiella pneumoniae BAA1706; Enterobacter aerogenes ATCC 13048; Serratia marcencens ATCC 14756; Providencia rettgeri BAA 2525; Pseudomonas aeruginosa 27853; Pseudomonas aeruginosa BAA 2108; and Acinetobacter baumannii BAA1709.

Preferably, the gram-positive bacterium may be a gram-positive coccus in grape-like clusters selected from a group consisting of: Staphylococcus aureus 29213; Staphylococcus aureus 43300; Staphylococcus aureus 700698; and Staphylococcus epidermidis 35984.

More preferably, the gram-positive bacterium may also be a gram-positive coccus in chain selected from a group consisting of: Enterococcus faecalis 29212; Enterococcus faecalis 51299; Enterococcus faecium 700221; Enterococcus caseiflavus 700668; and Enterococcus gallinarum 49608.

The bacterium may also be selected from a group consisting of: Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Haemophilus inkuenzae, Clostridium tetani (Tetanus), Clostridium perfringens, Clostridium botulinums, Cornynebacterium diphtheriae (Diphtheria), Legionella pneumophila, Coxiella burnetii, Brucella sp. (e.g., B. abortus, B. canis, B. melitensis, B. neotomae, B. ovis, B. suis and B. pinnipediae J Francisella sp. (e.g., F. novicida, F. philomiragia and F. tularensis), Streptococcus agalactiae, Neisseria gonorrhoeae, Chlamydia trachomatis, Treponema pallidum (Syphilis), Haemophilus ducreyi, Helicobacter pylori, Staphylococcus saprophyticus, Yersinia enterocolitica, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorferi, Porphyromonas. Streptococcus pneumonia, Mycobaterium tuberculosis and Heamophilus Influenzae.

In another embodiment, the microorganism may be a virus. The virus may be selected from a group consisting of: Orthomyxoviruses; Paramyxoviridae viruses; Metapneumovirus and Morbilliviruses; Pneumoviruses; Paramyxoviruses; Poxviridae; Metapneumoviruses; Morbilliviruses; Picornaviruses; Enteroviruseses; Bunyaviruses; Phlebovirus; Nairovirus; Heparnaviruses; Togaviruses; Alphavirus; Arterivirus; Flaviviruses; Pestiviruses; Hepadnaviruses; Rhabdoviruses; Caliciviridae; Coronaviruses; Retroviruses; Reoviruses; Parvoviruses; Delta hepatitis virus (HDV); Hepatitis E virus (HEV); Human Herpesviruses and Papovaviruses.

The Orthomyxoviruses may be Influenza A, B and C. The Paramyxoviridae virus may be Pneumoviruses (RSV), Paramyxoviruses (PIV). The Metapneumovirus may be Morbilliviruses (e.g., measles). The Pneumovirus may be Respiratory syncytial virus (RSV), Bovine respiratory syncytial virus, Pneumonia virus of mice, or Turkey rhinotracheitis virus. The Paramyxovirus may be Parainfuenza virus types 1-4 (PIV), Mumps, Sendai viruses, Simian virus 5, Bovine parainfuenza virus, Nipahvirus, Henipavirus or Newcastle disease virus. The Poxviridae may be Variola vera, for example Variola major and Variola minor. The Metapneumovirus may be human metapneumovirus (hMPV) or avian metapneumoviruses (aMPV). The Morbillivirus may be measles. The Picornaviruses may be Enteroviruses, Rhinoviruses, Heparnavirus, Parechovirus, Cardioviruses and Aphthoviruses. The Enteroviruses may be Poliovirus types 1, 2 or 3, Coxsackie A virus types 1 to 22 and 24, Coxsackie B virus types 1 to 6, Echovirus (ECHO) virus) types 1 to 9, ii to 27 and 29 to 34 or Enterovirus 68 to 71. The Bunyavirus may be California encephalitis virus. The Phlebovirus may be Rift Valley Fever virus. The Nairovirus may be Crimean-Congo hemorrhagic fever virus. The Heparnaviruses may be Hepatitis A virus (HAV). The Togaviruses may be Rubivirus. The Flavivirus may be Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus or Powassan encephalitis virus. The Pestivirus may be Bovine viral diarrhea (BVDV), Classical swine fever (CSFV) or Border disease (BDV). The Hepadnavirus may be Hepatitis B virus or Hepatitis C virus. The Rhabdovirus may be Lyssavirus (Rabies virus) or Vesiculovirus (VSV). The Caliciviridae may be Norwalk virus, or Norwalk-like Viruses, such as Hawaii Virus and Snow Mountain Virus. The Coronavirus may be SARS CoV-1, SARS-CoV-2, MERS, Human respiratory coronavirus, Avian infectious bronchitis (IBV), Mouse hepatitis virus (MHV), or Porcine transmissible gastroenteritis virus (TGEV). The Retrovirus may be Oncovirus, a Lentivirus or a Spumavirus. The Reovirus may be an Orthoreo virus, a Rotavirus, an Orbivirus, or a Coltivirus. The Parvovirus may be Parvovirus B 19. The Human Herpesvirus may be Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), or Human Herpesvirus 8 (HHV8). The Papovavirus may be Papilloma viruses, Polyomaviruses, Adenoviruess or Arenaviruses. Preferably, the virus is selected from the group consisting of SARS CoV, SARS CoV2, MERS or Influenza.

In another embodiment however, the microorganism may be a fungus. The fungus may be selected from the group consisting of Dermatophytres, including: Epidermophyton koccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme; or from Aspergillus fumigatus, Aspergillus kavus, Aspergillus niger, Aspergillus nidulans, Aspergillus terreus, Aspergillus sydowii, Aspergillus kavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp. Preferably, the fungi is selected from the group consisting of Aspergillus, Cryptococcus, or Pneumocystis.

In yet another embodiment the microorganism may be a protozoan. The protozoan may be selected from the group consisting of Entamoeba histolytica, Giardia lamblia, Cryptosporidium parvum, Cyclospora cayatanensis and Toxoplasma.

In one embodiment, the therapeutic agent is an antibiotic, an antiviral, an antifungal or an antiprotozoan agent. Preferably, the therapeutic agent is an antibiotic.

Preferably, the antimicrobial selection can be based on the bacterial gram staining, in line with the European Committee on Antimicrobial Susceptibility Testing (EUCAST) and the Clinical & laboratory Standard Institute (CLSI) protocols. Hence, the therapeutic agent is preferably an antimicrobial agent.

Accordingly, for gram-negative bacteria, the antimicrobial agent may be selected from a group consisting of: Amikacin; Gentamicin, Ciprofloxaxin, Imipenem, Meropenem, Ertapenem, Ceftazidime-avibactam, Piperacillin-tazobactam, Cefepime, Ceftazidime, Cefotaxime, Ceftazidime-clavulanic acid, Cefotaxime-clavulanic acid, Ceftalozane-tazobactam, Amoxacillin-clavulanic acid, Ampicillin, Fosfomycin, Nitrofurantoin, and Colistin.

For gram-positive bacteria, the antimicrobial agent may be selected from a group consisting of Penicillin, Ampicillin, cefoxitin, Oxacillin, Imipenem, Vancomycin, Linezolid, Gentamicin, Gentamicin high level, Levofloxacin, and Daptomycin.

Preferably, the antifungal agent may be selected from a group consisting of: caspofungin, micafungin, anidulafungin, posaconazole, voriconazole, flucytosine, amphotericine B, itraconazole, Posaconazole and fluconazole.

Preferably, the antiviral agent may be selected from a group consisting of: a protease inhibitor, such as ritonavir, atazanavir or darunavir; an inhibitor of viral DNA polymerase, such as acyclovir, tenofovir, valganciclovir or valacyclovir; and an inhibitor of integrase, such as raltegravir.

Preferably, the antiprotozoan agent may be selected from a group consisting of: metronidazole, atovaquone, benznidazole, dehydroemetine, eflornithine, emetine, fenbendazole, iodoquinol, melarsoprol, nifurtimox, pentamidine, quinacrine, sodium stilbogluconate, suramin, and tinidazole.

The fluorescent analysis used in the methods of the invention may be a flow cytometry analysis or a laser scanning analysis. The flow cytometer can be equipped with a plate reader for the automated analysis of each test reservoir. The flow cytometer can also be equipped with one blue laser and/or have 3 fluorescence channels.

In a preferred embodiment, the one or more flow cytometric parameters comprise forward scatter and/or side scatter and/or fluorescence parameters. The fluorescence scatter signal may be intensity, spectral profile and/or cell count.

The fluorescent marker may be selected from a group consisting of: a nucleic acid stain; a metabolic stain; a membrane potential stain; a probe for organelles; fluorescent tracer of cell morphology and fluid flow; probe for cell viability; proliferation and function; and/or probe for reactive oxygen species.

Preferably, the fluorescent marker may be selected from the group consisting of: acridine dye; cyanine dye; fluorone dye; oxazin dye; phenanthridine dye; or a rhodamine dye. Most preferably, the fluorescent marker may be selected from the group consisting of: CTC (5-Cyano-2,3-ditolyl tetrazolium chloride), Calcein AM, Dihydrorhodamine 123, DIBAC₄(3), DioC 2(3), Fluorescein Diacetate, 5CFDA, AM, CFDA-SE, Propidium Iodine, SYTO 16 Green Fluorescent, and Nucleic Acid Stain.

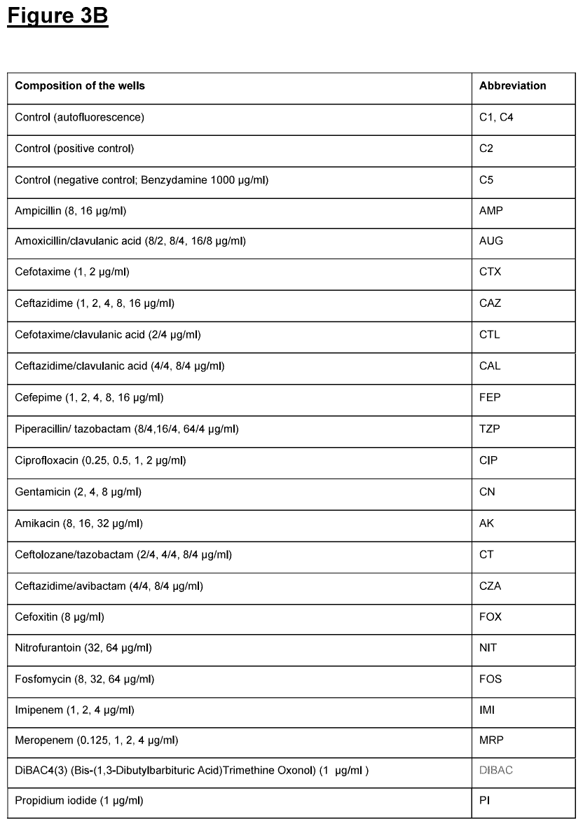

In a preferred embodiment, the fluorescent marker DiBAC₄(3) is combined with the antibiotics as described in FIG. 3A for testing the susceptibility of gram-negative bacteria to these antibiotics.

Preferably, the fluorescent marker propidium iodide (PI) is combined with the antibiotic imipenem for testing Pseudomonas spp's susceptibility to imipenem.

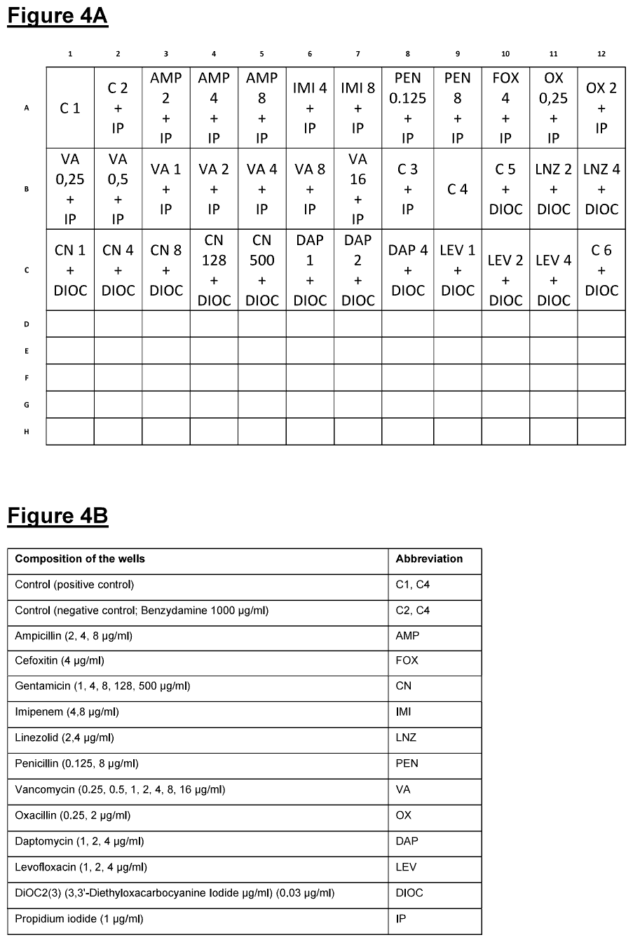

Most preferably, the fluorescent marker propidium iodide (PI) may be combined with the antibiotics as described in FIG. 4A for testing the susceptibility of gram-positive bacteria to these antibiotics.

The fluorescent marker DiOC2(3) (3,3′-Diethyloxacarbocyanine Iodide) may be combined with the antibiotics as described in FIG. 4A for testing the susceptibility of gram-positive bacteria to these antibiotics.

Preferably, the fluorescent marker propidium iodide (PI) may be combined with colistin for testing the susceptibility of gram-positive bacteria to colistin.

In one embodiment, the methods of the invention may comprise at least one set of:
(i) the one or more first test reservoir containing a fluorescent marker/therapeutic agent combination,
(ii) the one or more second positive control reservoir, and
(iii) the one or more third negative control reservoir both containing the same fluorescent marker, and optionally
(iv) the one or more fourth auto-fluorescent control reservoir in which neither the fluorescent marker or therapeutic agent are present.

In other words, preferably at least one combination of fluorescent marker/therapeutic agent (preferably, fluorescent marker/antibiotic) are tested. In this set, the fluorescent marker is the same.

In another embodiment, the method of the first aspect comprises a plurality of sets of:
(i) the one or more first test reservoir containing a fluorescent marker/therapeutic agent combination,
(ii) the one or more second positive control reservoir, and
(iii) the one or more third negative control reservoir both containing the same fluorescent marker, and optionally
(iv) the one or more fourth auto-fluorescent control reservoir in which neither the fluorescent marker or therapeutic agent are present.

In other words, preferably at least two, three, four or more different combinations of fluorescent marker/therapeutic agent (preferably, fluorescent marker/antibiotic) are tested. Preferably at least five, six, seven or more different combinations of fluorescent marker/therapeutic agent (preferably, fluorescent marker/antibiotic) are tested. Within in each set, the fluorescent marker is the same. However, the fluorescent marker may be the same or different between the plurality of sets. The number and type of combinations of fluorescent marker/therapeutic agent may depend on whether gram-negative or gram-positive bacteria are being tested for.

The reservoir may be a container, a tube or a well. Preferably, the reservoir is a well. For example, the well may be part of a plate, such as a 96-well plate.

In a second aspect of the invention, there is provided a kit for use in the method of the first aspect.

The kit of the present invention may be a panel comprising the one or more first, second, the third, and/or the fourth reservoirs. Preferably, the panel can be a 96-well plate.

Advantageously, the inventors have carefully designed testing panels that are optimised for the detection of variety of microorganisms. Each testing panel comprises carefully selected combinations of therapeutic agent/antibiotic and fluorescent markers, in which each therapeutic agent/antibiotic and fluorescent marker/fluorochrome combination has been optimised to increase the accuracy of the readouts. The invention represents the first disclosure of pre-set combinations of drugs and fluorochromes in a testing panel before incubating the microorganism.

In one embodiment, the panel may be the FASTgramneg panel disclosed in FIG. 3A. Preferably, the FASTgramneg panel comprises the wells as disclosed in FIG. 3B. Thus, preferably the panel is configured to analyse gram-negative bacteria. Preferably, in this panel for analyzing gram-negative bacteria, the antibiotics Ampicillin, Amoxacillin-clavulanic acid, cefotaxime, ceftazidime, cefotaxime-clavulanic acid, ceftazidime-clavulanic acid, ceftalozane-tazobactan, cefepime, cefoxitin, ceftazidime-avibactam, piperacillin-tazobactam, ciprofloxacin, gentamicin, amikacin, nitrofurantoin, fosfomicin, imipenem and meropenem are preferably combined with DIBAC. Preferably, imipenem is combined with PI (in another well).

In another embodiment, the panel may be the FASTgrampos panel disclosed in FIG. 4A. Preferably, the FASTgrampos panel comprises the wells as disclosed in FIG. 4B. Preferably, the panel, therefore, is configured to analyse gram-positive bacteria. Preferably, in this panel for analyzing gram-positive bacteria, the antibiotics Ampicillin, cefoxitin, oxacillin, penicillin, vancomycin and imipenem are preferably combined with PI. Preferably, Gentamicin, linezolid, daptomycin, levofloxacin are combined with DIOC.

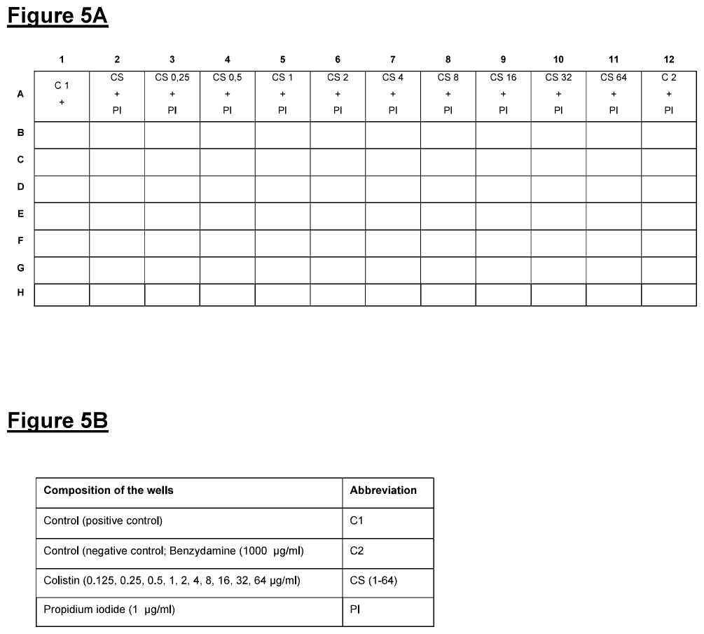

In yet another embodiment, the panel may be the FASTcolistin MIC panel disclosed in FIG. 5A. Preferably, the FASTcolistin MIC panel comprises the wells as disclosed in FIG. 5B. Preferably, the panel, therefore, is configured to analyse gram-negative bacteria. Preferably, in this panel for analysing gram-negative bacteria, Colistin MIC is combined with PI.

The inventors believe that the method of purification of the microorganism described above is novel. As described above, the present disclosure constitutes the first account of the use of a density gradient solution (e.g. Histopaque®) in a microbiological assay for purifying, identifying and/or diagnosing microorganisms. The inventors have demonstrated that this method is surprisingly much more effective than the standard physical separation used in the prior art, as there is no loss of cells during centrifugation and the suspension is much cleaner, and therefore more pure.

Therefore, in a third aspect of the invention, there is provided a method of purifying a microorganism from a biological sample comprising:
(i) obtaining a biological sample comprising a microorganism; and
(ii) contacting the biological sample with a density gradient solution, to thereby purify the microorganism.

In a fourth aspect, therefore, there is provided a use of a density gradient solution, for purifying a microorganism from a biological sample.

The microorganism may be purified in the biological sample. The method may comprise a subsequent step of extracting the microorganism from the biological sample.

Preferably, the density gradient solution is Histopaque®- 1077). Histopaque™ is a density gradient cell separation medium comprising Ficoll and sodium diatrizoate. Most preferably, the biological sample and the density gradient are present at a ratio of 1:1.

After step contacting the biological sample with a density gradient solution, the sample preparation step then preferably comprises centrifuging the sample, preferably for at least one minute, preferably at about 13,000 rpm. The sample preparation step comprises re-suspending the resulting in pellet (comprising purified microorganisms) in media. The media may preferably be cation-adjusted broth, optionally about 1 ml of sterile and filtered Mueller Hinton II cation-adjusted broth.

The method preferably comprises contacting the biological sample with a haemolytic agent before contacting the biological sample with the density gradient solution. Preferably, the haemolytic agent is configured to lyse any contaminating cells in the sample, such as contaminating host cells emanating from the host from whom the sample is taken, or debris present in environmental samples. The use of the haemolytic agent is preferred in embodiments in which the sample comprises blood, preferably a blood culture. Protocol B disclosed in FIG. 1 illustrates one preferred embodiment of the sample preparation step of the method, when the biological sample is a blood sample.

In one embodiment, the haemolytic agent may be Triton™ X-100 (commonly called Triton X-100). Triton™ X-100 is a common non-ionic surfactant and emulsifier which is often used in biochemical applications to solubilize proteins. It is considered a comparatively mild and non-denaturing detergent. It is utilised for lysing cells to extract protein and cellular organelles. It can also permeabilise a living cell membrane for transfection, or used for DNA extraction.

Preferably, Triton X-100 is used at a concentration of between 0.1% and 3% (v/v), or between 0.2% and 2.5% (v/v) or between 0.3% and 2% (v/v), or between 0.4 and 1% (v/v), and preferably about 0.5% (v/v). Preferably, the biological sample is contacted with the haemolytic agent (preferably Triton X-100) for at least 1 minute, 2 minutes or 5 minutes. Preferably, the biological sample is contacted with the haemolytic agent (preferably Triton X-100) for less than 2 hours, 1 hour, 30 minutes or less than 15 minutes. This step may be carried out at room temperature. Thus, preferably the same preparation step comprises contacting the biological sample with a haemolytic agent for five minutes at room temperature.

After contacting the biological sample with a haemolytic agent and before contacting the same with the density gradient solution, the sample preparation step preferably comprises centrifuging the sample, preferably for at least one minute, preferably at about 13,000 rpm. Preferably, the sample preparation step then comprises re-suspending the resulting pellet (with the microorganisms). Preferably, the pellet is re-suspended in saline solution, optionally about 0.05 ml to 2 ml, 0.01 ml to 1 ml, or 0.25 ml to 0.75 ml of sterile and filtered saline solution. Most preferably, the pellet is re-suspended in 0.5 ml of sterile and filtered saline solution.

The haemolytic agent used in the extraction method allows the lysing of the host's cells, in particular red blood cells, and any potential debris that may present in the biological sample, while preserving the microorganism's cells. The method described herein allows the purified microorganisms to accumulate towards the bottom of the tube, while the cell debris and host's cells combined with density gradient solution accumulate towards the top of the tube. Preferably, the method comprises extracting the microorganism from the sample.

The method may further comprise identifying the microorganism before introducing the biological sample comprising the microorganism into each of the first, second, third and/or fourth reservoir as defined in the first aspect. The identification of the microorganism may be performed using genetic methods, microarrays, physical methods and/or mass spectrometry methods. Genetics methods may be a quantitative Polymerase Chain Reaction (PCR), an immuno-PCR, or a combination thereof. Microarrays may be DNA microarrays, protein microarrays, antibody microarrays, or a combination thereof. Physical methods may be an infrared and Raman spectroscopy or a laser-induced breakdown spectroscopy (LIBS). Mass spectrometry methods may be performed through an ICP mass spectrometer, DART mass spectrometer, or a MALDI-TOF Brucker.

Thus, in a preferred embodiment, the extraction method of the first aspect comprises:
(i) obtaining a biological sample comprising a microorganism;
(ii) contacting the sample with a density gradient solution;
(iii) centrifuging the sample; and
(iv) re-suspending the resulting in pellet.

Preferably, the extraction method further comprises, between step (i) and (ii):
(a) contacting the biological sample with a haemolytic agent;
(b) centrifuging the sample; and
(c) optionally re-suspending the resulting pellet.

Thus, in a further aspect, there is provided a method for determining the susceptibility phenotype, to at least one therapeutic agent, of a microorganism present in a biological sample, the method comprising:
(i) a sample preparation step comprising:
   (a) obtaining the biological sample comprising the microorganism;
   (b) contacting the sample with a density gradient solution; and
   (c) optionally identifying the microorganism;
(ii) introducing a biological sample comprising a microorganism into each of: (d) one or more of a first, test reservoir,
   (e) one or more of a second, positive control reservoir, and
   (f) one or more of a third, negative control reservoir, wherein the negative control reservoir comprises non-viable microorganisms;
   (g) one or more of a fourth auto-fluorescence control reservoir,
(iii) contacting the biological sample in the one or more first, test reservoir with at least one therapeutic agent;
(iv) contacting the biological sample in the one or more first, second and third reservoir with at least one fluorescent marker; and
(v) performing a fluorescence analysis in order to obtain one or more fluorescence parameters for the biological sample in each of the reservoirs,
   wherein the microorganism's susceptibility phenotype to the at least one therapeutic agent is obtained by comparing one or more fluorescence parameters between the reservoirs.

Preferably, the density gradient solution is Histopaque®.

Preferably, the extraction method of the first aspect further comprises between step (a) and (b):
(i) contacting the biological sample with a haemolytic agent.

In one embodiment, the haemolytic agent may be incubated with the biological sample for less than or at least 10 minutes, less than or at least 9 minutes, less than or at least 8 minutes, less than or at least 7 minutes, less than or at least 6 minutes, less than or at least 5 minutes, less than or at least 4 minutes, less than or at least 3 minutes, less than or at least 2 minutes or less than or at least 1 minute.

In another embodiment, the haemolytic agent may be incubated with the biological sample for less than 30 seconds, 20 seconds, 10 seconds or 5 seconds. Preferably, the haemolytic agent may be incubated with the biological sample for less than 4 s, 3 s, 2 s or 1 s.

Preferably, the haemolytic agent is Triton-X100.

In another and preferred embodiment, the haemolytic agent is Tergitol.

Preferably, Tergitol is used at a concentration of 10%. Preferably, the biological sample is contacted with Tergitol, and mixed (preferably by vortex).

All of the features described herein (including any accompanying claims, abstracts and drawings), and/or all of the steps of any method or process so disclosed, may be combined with any of the above aspects in any combination, except combinations where at least some features and/or steps are mutually exclusive.

For a better understanding of the invention, and to show how embodiments of the same may be carried into effect, reference will now be made, by way of example, to the accompanying Figures, in which:—

FIG. 1 shows a schematic representation of two embodiments of a sample extraction protocol (Protocol A and Protocol B).

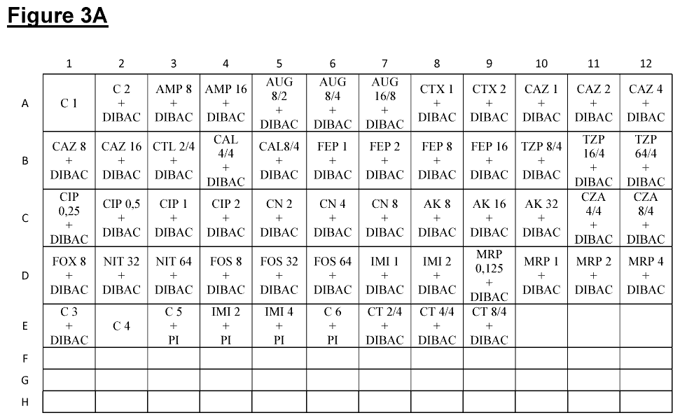

FIG. 2 is a summary table of the chemicals tested to kill the cells contained in the negative control for a fluorescent probe used in an embodiment of the method of the invention.

FIG. 3A shows the layout of one embodiment of a FASTgramneg panel according to the invention for testing Gram-negative bacteria.

FIG. 3B shows a summary table of the composition of each well of the FASTgramneg panel shown in FIG. 3A.

FIG. 4A discloses the layout of one embodiment of a FASTgrampos panel according to the invention for testing Gram-positive bacteria.

FIG. 4B shows a summary table of the composition of each well of the FASTgrampos panel shown in FIG. 4A.

FIG. 5A discloses the layout of one embodiment of a FASTcolistin MIC panel according to the invention for testing Gram-negative bacteria.

FIG. 5B shows a summary table of the composition of each well of the FASTcolistin MIC panel shown in FIG. 5A.

FIG. 6A shows the number of Gram-negative bacteria in each species, correctly identified on MALDI-TOF from blood culture samples purified through the sample preparation step of the invention, compared to those obtained from colonies forming units derived from overnight (24 hours) subcultures of blood samples.

FIG. 6B shows the number of Gram-positive bacteria in each species, correctly identified on MALDI-TOF from blood culture samples purified through the sample preparation step of the invention, compared to those obtained from colonies forming units derived from overnight (24 hours) subcultures of blood samples.

### EXAMPLES

The inventors have developed a novel method or assay for determining the susceptibility phenotype of a microorganism (such as a bacterium, fungus, virus or protozoan) from a biological sample to at least one therapeutic agent, such as an antibiotic. The method is referred to as the “FASTinov susceptibility assay”. The inventors have also developed a kit specifically designed to evaluate, by flow cytometry, the antimicrobial susceptibility to several antibiotics, which is called the “FASTinov panel”. As part of the sample preparation steps for use in the assay and kit, different methods of microorganism extraction and purification were tested in order to optimise the sample purity for subsequent analysis on the panel. In the process, the inventors have developed an optimised protocol which guarantees an optimal sample quality and purity.

To assess the robustness of their method, the inventors tested two different blood samples spiked with one each time of the thirty of the most common bacteria isolates generally found in clinical isolates (from the FASTinov collection). Depending on the isolates under analysis, an optimised combination of fluorescent probe and antibiotic were used.

The inventors further designed an innovative panel layout comprising the different optimised features of the present invention.

### Materials and Methods

### (i) Sample Preparation

### Microorganisms

Thirty bacteria belonging to the FASTinov collection and representing the most common bacterial isolates found in clinical settings were selected for this study. These isolates consisted of a both ATCC and clinical strains. Of those, ten isolates were gram-negative bacilli ten were gram-positive cocci in grape-like cluster (such as Staphylococcus spp) and ten were gram-positive cocci chains (Enterococcus spp).

### Samples Studied

Two types of blood culture were studied:
(i) the Bactec PLUS Aerobic/F, ref 442192 (AR), and
(ii) the LYTIC/10 ANAEROBIC/F ref 442265 (ANA) from Becton Dickinson, S. A

Both blood cultures were spiked (i.e. infected) with the selected bacteria at FASTinov laboratories in Porto (Portugal) according to the protocol described in [2] with slight modifications as detailed below.

Previously frozen isolates were sub-cultured to prepare bacterial suspensions. The bacteria were grown overnight in Mueller-Hinton agar and the concentration was adjusted in phosphate buffered saline (PBS). For this purpose, aerobic blood culture bottles were spiked with 2×10³ bacterial cells/bottle along with 8 mL of whole-blood samples obtained from blood donors. The inoculated blood cultures (BC) were incubated on the recommended BD equipment until they flagged positive. Blood culture bottles from BioMérieux were also tested. Urine samples from heathy donors were also inoculated [15] with the same strains and submitted to the same protocol.

Extraction of the Microorganisms from the Samples

To start the AST using the novel FASTinov technology, a purified bacteria suspension obtained from clinical or veterinary samples was required. Referring to FIG. 1, two different protocols (Protocol A and Protocol B) for extracting the bacteria from positive (i.e. infected) blood cultures were developed and the number of viable cells recovered was compared:

### 1) Protocol A

After vortex-agitation of the blood culture, a vacuum blood collection gel tube (BD Vacutainer® Barricor™ Blood Collection Tube, ref 365056) was filled with about 5.5 ml and centrifuged at 1500 rpm for 5 minutes. The supernatant was discarded and the pellet was resuspended in 1 ml of sterile and filtered (through a 0.22 micrometres filter) saline solution. One (1) ml of the suspension was transferred to an Eppendorf tube and centrifuged at 13,000×rpm (11337 g) during 1 min; the supernatant was then discarded. One (1) ml of sterile and filtered (through a 0.22 micrometres filter) Muller-Hinton cation adjusted broth (ref BD 275730) was added to the Eppendorf tube and vortex-agitated. Colony forming units (CFUs) were assessed subsequently. This process was carried out in duplicate.

### 2) Protocol B

After vigorous vortex-agitation of the blood culture, a vacuum blood collection gel tube, for example a 2 ml tube (Tube A in FIG. 1), was filled with the culture without any additives. A lysing agent, 50 μl of Triton X-100 at 10% (v/v), was then added to 1 ml of the contents of the collection tube, vortex-agitated and incubated for 5 minutes at room temperature. The mixture was then centrifuged at preferably 13000 rpm for 1 min, the supernatant was discarded and the pellet was resuspended in 0.5 ml of sterile and filtered (through a 0.22 micrometres filter) saline solution. Histopaque®-1077 (ref 10771) was added to a new tube (Tube B in FIG. 1), and the resuspended lysed sample was added to the Histopaque; preferably 0.5 ml of the lysed sample was added to 0.5 ml of Histopaque®-1077 already into a tube, and centrifugation was repeated at preferably 13000 rpm for about 1 min; the supernatant was discarded. One (1) ml of sterile and filtered (through a 0.22 micrometres filter) Mueller Hinton II cation adjusted broth (ref BD 275730) was added to the Eppendorf tube and vortex-agitated. Colony forming units (CFUs) were assessed subsequently. This process was carried out in duplicate.

### Statistical Analysis

Differences between the CFUs obtained from aerobic (AR) and anaerobic (ANA) blood culture (BC) bottles after extraction the microorganisms with protocol A, and using AR bottles protocol A and B were evaluated using Wilcoxon signed rank test. All the experiments were performed in duplicate and a mean value calculated. Significant differences were considered with a p-value below 0.05 (<0.05). The statistical analysis was performed using IBM SPSS statistics version 24.0.

### Identification of the Infectious Agent

The suspension obtained after treatment with TRITON X-100 and Histopaque was used to identify the microorganisms by MALDI-TOF (Bruker) using the septityper mode. Alternatively, the sample purified via Protocol B was further centrifuged at or 13000 rpm to 15000 rpm for 1 minute. The resulting pellet was then dried at room temperature or at 37° C., before inoculating the MALDI-TOF (Bruker). The accuracy of the detection obtained using the dried pellet was compared to that of overnight cultures.

### (ii) FASTinov Panel Specifications

### Optimisation of the Different Panel Elements and Processing Times

The inventors designed an innovative panel which is used to evaluate the antimicrobial susceptibility of different types of bacteria to several antibiotics by flow cytometry. The optimisation of the antibiotic/probe combination for selected groups of bacteria, such as Enterobacterales, Pseudomonas, Acinetobacter, Staphylococcus or Enterococcus was performed, and resulted in a layout of a 96-well plate panel for gram-positive bacteria (FASTgrampos—see FIGS. 4A and 4B) and a 96-well plate panel for gram-negative bacteria (FASTgramneg—see FIGS. 3A and 3B). The inventors very carefully selected the probe/concentration/time that optimally separated susceptible from resistant strains amongst a wide variety of fluorescent probes available on the market.

For the detection of some mechanisms of resistance, the Gram-negative panel includes some drugs, such as ceftazidime-clavulanic acid and cefotaxime-clavulanic acid for ESBL; cefoxitin for AmpC screening and a low value of meropenem (0.25 ug/ml) for screening for carbapenemases.

As the Gram-positive panel includes several concentrations of vancomycin, it is possible to determine both the MIC values (minimal inhibitory concentration) by flow cytometry and the phenotype.

Regarding colistin sensitivity, a separate panel was designed to provide the phenotype and the MIC for Enterobacterales, Pseudomonas and Acinetobacter spp. The FASTcolistin MIC panel is shown in FIGS. 5A and 5B. The antibiotic sequences and their order of appearance on the FASTgrampos and FASTgramneg panels were strategically developed by the inventors. Furthermore, the inventors implemented an optimal washing between each well aiming to reduce the carryover effect identified during development stages. If left unmanaged, this carryover effect could generate detrimental results such as false susceptibility, which in turn would induce major errors. Furthermore, two controls were included on the panels in order to ensure effective susceptibility readouts: (i) a positive control (non-treated cells) which is commonly required in microbiology antimicrobial susceptibility assays, and (ii) a negative control (dead cells) to control the viability of the probe, which is typically used in cytometry assays, but not in microbiology assays. This novel addition confers an innovative twist to the new inventors' panel (see FIG. 2).

Furthermore, these controls ensure that only viable (and not non-viable) microorganisms are considered in the susceptibility assessment. Non-viable microorganisms cannot respond to therapeutic agents. If they were to be included in the susceptibility assessment, a microorganism could wrongly appear resistant to all therapeutic agents, which would correspond to a false negative/diagnostic and “major error”, and consequently, result in the implementation of the wrong therapy. Likewise, if the fluorescent probe is not present or is not performing well, the effect of the therapeutic agent on the microorganism will be difficult or impossible to assess. Therefore, the addition of the novel negative control significantly enhances the accuracy of AST.

(iii) Running AST Using the Newly Designed Panels

In order to run the AST using the new panels, the inventors have implemented the following steps:

### Microorganism Selection

A substantial amount of ATCC bacteria were selected for this study. Fifteen (15) gram-negative bacilli including Escherichia coli ATCC 25922, Escherichia coli ATCC 8739, Escherichia coli ATCC 35218, Escherichia coli BAA 2425, Klebsiella pneumoniae ATCC 13443, Klebsiella pneumoniae BAA 1705, Klebsiella pneumoniae ATCC 700603, Klebsiella pneumoniae BAA1706, Enterobacter aerogenes ATCC 13048, Serratia marcencens ATCC 14756, Providencia rettgeri BAA 2525, Pseudomonas aeruginosa 27853, Pseudomonas aeruginosa BAA 2108, Acinetobacter baumannii BAA1709 were selected. An additional ten (10) gram-positive cocci in grape in grape-like cluster including Staphylococcus aureus 29213, Staphylococcus aureus 43300, Staphylococcus aureus 700698, Staphylococcus epidermidis 35984); and five (5) gram-positive cocci chains including Enterococcus faecalis 29212, Enterococcus faecalis 51299, Enterococcus faecium 700221, Enterococcus caseiflavus 700668, Enterococcus gallinarum 49608) were selected. These bacteria represent the most important and common bacteria found in clinical settings. Tables 1, 2 and 4 below summarise the distribution of bacteria on each panel.

TABLE 1
Distribution by species of the Gram-negative
bacteria studied on FASTgramneg kit

| | Total Blood cultures |
|---|---|
| Enterobacterales | 152 |
| E. coli | 64 |
| Kl. pneumoniae | 54 |
| Kl. oxytoca | 2 |
| Kl. aerogenes | 1 |
| Serratia marcencens | 3 |
| Providencia rettegeri | 1 |
| Proteus mirabilis | 9 |
| Enterobacter cloacae | 12 |
| Enterobacter aerogenes | 4 |
| Enterobacter kobei | 1 |
| Citrobacter koseri | 2 |
| Citrobacter freundii | 1 |
| Non-fermentors | 109 |
| Ps. aeruginosa | 77 |
| Acinetobacter baumannii | 30 |
| Acinetobacter calcoaceticus | 1 |
| Acinetobacter pitti | 1 |
| Total Gram-negative bacilli | 261 |

TABLE 2
Distribution by species of the Gram-positive
bacteria studied on FASTgrampos kit

| | Total Blood cultures |
|---|---|
| Sphaphylococcus spp | 112 |
| S. aureus | 47 |
| S. epidermidis | 47 |
| S. hominis | 15 |
| S. capitis | 1 |
| S. lugdunensis | 1 |
| S. haemolyticus | 1 |
| Enterococcus spp | 87 |
| E. faecalis | 54 |
| E. faecium | 27 |
| E. gallinarum | 4 |
| E. casseiflavus | 1 |
| E. raffinosus | 1 |
| Total Gram-positive | 199 |

TABLE 3
Distribution by species of the Gram-negative
bacteria studied on FASTcolistin MIC kit

| | Total Blood cultures |
|---|---|
| Enterobacterales | 131 |
| E. coli | 24 |
| Kl. pneumoniae | 60 |
| Kl. aerogenes | 2 |
| Serratia marcencens | 3 |
| Providencia rettegeri | 1 |
| Proteus mirabilis | 4 |
| Enterobacter cloacae | 24 |
| Enterobacter aerogenes | 1 |
| Enterobacter kobei | 1 |
| Enterobacter hormaechei | 1 |
| Citrobacter koseri | 1 |
| Citrobacter freundii | 5 |
| Morganella morganii | 4 |
| Non-fermentors | 123 |
| Ps. aeruginosa | 59 |
| Acinetobacter baumannii | 64 |
| Total Gram-negative bacilli | 254 |

### Samples Analysed

Blood cultures (Bactec PLUS Aerobic/F, ref 442192 from Becton Dickinson, S. A) were spiked with selected bacteria at FASTinov laboratories in Porto (Portugal) according to the protocol described in [2] with slight modifications as detailed earlier. The inoculated blood cultures (BC) were incubated on the recommended BD equipment until they flagged positive. Urine samples were inoculated with the same strains and submitted to the same protocol thus bringing urinary tract infection diagnostics under the potential uses of newly designed panels for a large array of antibiotics.

### Drug/Fluorochrome Combinations

The most important antimicrobials at breakpoint concentrations based on the EUCAST and CLSI protocols were selected according to a microorganism identification pattern (see Tables 4 and 5 below), for testing on the different samples, preferably on the blood cultures and urine samples prepared as detailed above. Different fluorescent probes at different concentrations were tested; the lower concentration of each probe that showed the best discrimination between untreated and dead cells (indicative of being susceptible) were selected.

TABLE 4
Table of drugs included on FASTgramneg
per group of microorganisms

| | Enterobacterales | | Pseudomonas spp | | Acinetobacter spp | |
|---|---|---|---|---|---|---|
| Antibiotics: | EUCAST | CLSI | EUCAST | CLSI | EUCAST | CLSI |
| Amikacin | ✓ | ✓ | ✓ | ✓ | ✓ | ✓ |
| Gentamicin | ✓ | ✓ | | ✓ | ✓ | ✓ |
| Ciprofloxacin | ✓ | ✓ | ✓ | ✓ | ✓ | ✓ |
| Imipenem | ✓ | ✓ | ✓ | ✓ | ✓ | ✓ |
| Meropenem | ✓ | ✓ | | | | |
| Ceftazidime- | ✓ | ✓ | ✓ | ✓ | | |
| avibactam | | | | | | |
| Piperacillin- | ✓ | ✓ | ✓ | ✓ | | ✓ |
| tazobactam | | | | | | |
| Cefepime | ✓ | ✓ | ✓ | ✓ | | |
| Ceftazidime | ✓ | ✓ | ✓ | ✓ | | |
| Cefotaxime # | ✓ | ✓ | | | | |
| Ceftazidimie- | ✓ | ✓ | | | | |
| clavulanic | | | | | | |
| acid # | | | | | | |
| Cefotaxime - | ✓ | ✓ | | | | |
| Clavulanic | | | | | | |
| acid # | | | | | | |
| Ceftalozane- | ✓ | ✓ | ✓ | ✓ | ✓ | ✓ |
| tazobactam | | | | | | |
| Amoxacillin- | ✓ | ✓ | | | | |
| clavulanic acid | | | | | | |
| Ampicillin | ✓ | ✓ | | | | |
| Fostomycin | ✓ | ✓ | | | | |
| Nitrofuran- | ✓ | ✓ | | | | |
| toin * | | | | | | |
| Colistin | ✓ | ✓ | ✓ | ✓ | ✓ | ✓ |

Detection of mechanisms of resistance:
ESBL (Enterobacterales group I)
Screening for the presence of:
ESBL (Enterobacteroles group II)
pAmpC
carbapenemases
* Regarding Enterobacterales, only for E. coli according EUCAST and CLSI protocol
# For determination of mechanisms of resistance

TABLE 5
Table of drugs included on FASTgrampos
per group of microorganisms

| | Staphylococcus spp | | Enterococcus spp | |
|---|---|---|---|---|
| Antibiotics | EUCAST | CLSI | EUCAST | CLSI |
| Penicillin* | ✓ | ✓ | ✓ | ✓ |
| Ampicillin | | | ✓ | ✓ |
| Cefoxitin** | ✓ | ✓ | | |
| Oxacillin*** | ✓ | ✓ | | |
| Imipenem | | | ✓ | ✓ |
| Vancomycin**** # | ✓ | ✓ | ✓ | ✓ |
| Linezolid | ✓ | ✓ | ✓ | ✓ |
| Gentamicin | ✓ | ✓ | | |
| Gentamicin high level # | | | ✓ | ✓ |
| Levofloxacin | ✓ | ✓ | ✓ | ✓ |
| Daptomycin | ✓ | ✓ | ✓ | ✓ |

*Regarding Staphylococcus, only for S. aureus according EUCAST protocol
**Not aplicable to S. epidermidis
***Only for S. epidermidis
****MIC's value for S. aureus
# regarding Enterococcus only for E. faecalis

### Controls

An auto-fluorescence control well (untreated and unstained cells), a positive control or viability control well (non-treated and stained cells with each fluorescent probe), and a negative control well (stained dead cells, accounting for a positive control for the fluorescent dye) were thoughtfully distributed in the panel in order to ensure the viability of the strain and activity of the probe. Different methods for a negative control were tested at different concentrations, stained with different probes and compared on flow cytometry analysis (see FIGS. 3-5).

### Specific Layout of the Panels

The panels include a first well for autofluorescence control, followed by the positive control well, stained with the probe without any antibiotic. The antibiotics were always put on the panel from a lower concentration to a higher concentration and combined with the probe. In the flow cytometer acquisition template, a three (3) second backflush was scheduled between analysed wells of the same drug; and a twenty (20) second backflush programmed between wells of different drugs. Each well was individually mixed for three (3) seconds. Drugs (e.g. antibiotics) that showed the highest intensity of fluorescence when incubated with susceptible strains, were deliberately positioned at the end of the panel. For example, given that FASTgrampos panel has two different fluorescent probes, a second autofluorescence well and a control stained with the second probe were included before the drugs are stained with the second probe (see one of the panel displayed in FIGS. 3-5 of panel layout). Carryover studies were undertaken to ascertain that specificity of fluorescence readouts corresponded to actually marked cells instead on unspecific or background fluorescence signal (see FIGS. 3-5).

### Dried Panel Performance and Controls

All of the optimisations were performed on freshly produced panels and, in order to obtain a product that is stable at room temperature, lyophilisation and the drying process were tested. On the sample that was stable at room temperature, antimicrobial drugs were quantified using both micro-dilution assay and HPLC, and the fluorescent activity was evaluated by flow cytometry. Real time stability studies were performed.

### Inoculation and Incubation of the Panels

After sample preparation, a bacterial suspension with a MacFarland optical concentration of 0.5 was prepared in sterile saline solution and diluted on Mueller Hinton II cation adjusted broth (BD 275730). Cell concentration between 1×10⁶ to 1×10⁷ cell/ml were considered the optimal value. The cells were incubated for 1 hour at 37° C. with shaking.

### Flow Cytometer Analysis

For this analysis, the flow cytometer had the following specifications: equipped with one blue laser (488 nm; output: 50 mW; beam spot size: 5×80 μm);
had 3 fluorescence channels: 525/40 BP, 585/42 BP and 690/50 BP; and
equipped with a plate reader for the automated analysis of each panel.

Setting optimizations were performed for Enterobacterales, Acinetobacter spp, Pseudomonas spp, Staphylococcus spp and Enterococcus spp. Parameters, such as the intensity of fluorescence of the cells, the number of cells acquired by well and the cells light scatter information were recorded, and cells treated with antibiotics, were systematically compared to untreated cells (positive control). Using the inventors' (FASTinov) templates of analysis, the panels were analysed, data recorded and a report generated using bioFast software, which includes a proprietary algorithm. Validation criteria were thoughtfully included such as viability of the strain (positive control) and activity of the probe (negative control). Additionally, the number of events on the optimised gate (zone of analysis where the bacteria are represented) was also specifically included as a validation criteria. A minimum number of cells is needed to validate the assay.

### Reference Method

In order to evaluate the phenotypic results obtained from flow cytometry analysis, disk diffusion method was used for all isolates to determine the antimicrobial susceptibility as described in the reference protocols from EUCAST [3] and CLSI [4]. For Colistin (on gram-negative bacteria) and vancomycin (in the case of Staphylococcus), the method used to determine the antimicrobial susceptibility was broth microdilution [5]. Broth microdilution was also performed for meropenem as there is a need to know if the MIC is higher than the 0.25 ug/ml required for the possibility for the screening of carbapenemases [6]. Standard methodologies were performed from the same inoculum used to spike the blood cultures. Quality control of the reference methods was also performed according to the recommendations of EUCAST and CLSI.

### Data Analysis

The susceptible (S), intermediate (I) and resistant® results obtained with the FASTinov® test were compared to the reference disk diffusion method and/or with MIC values determined by micro-dilution.

### (iv) Performance Evaluation

### Validation Sites

Two sites were used for validation of the different FASTinov® kits (FASTgrampos, FASTgramneg and FASTcolistin MIC) directly from positive blood cultures in FASTinov laboratories in Porto (Portugal), using spiked blood cultures and at hospital Ramon y Cajal in Madrid (Spain) with patients' blood cultures.

### Bacterial Strains

A total of 256 gram-negative bacilli (180 at FASTinov and 81 at Ramon y Cajal hospital) were studied on the FASTgramneg kit, and 199 Gram-positive bacteria (131 at FASTinov and 68 at Ramon y Cajal hospital) on the FASTgrampos kit. Different species of Enterobacterales, Pseudomonas and Acinetobacter were studied, as well as Staphylococcus and Enterococcus. The FASTcolistinMIC was performed at both FASTinov and Ramon y Cajal hospital.

### Antimicrobial Susceptibility Assay Using Reference Method

All the strains were classified as S, I or R according the reference methods. MIC was determined for vancomycin and colistin. The presence of ESBL on Enterobacterales group I, together with the screening for ESBL on Enterobacterales group II, and the screening for AmpC and presence of carbapenemases according EUCAST protocol for detection of mechanisms of resistance were also performed.

### Antimicrobial Susceptibility Assay Using FASTinov Kits (FASTgramneg, FASTgrampos and FASTcolistin MIC)

Following the sample preparation protocol optimised by the inventors (FASTinov) namely protocol B for bacteria extraction from the blood culture described above, the inoculation of the samples on the newly designed FASTinov panels and flow cytometric analysis, the antimicrobial susceptibility results were obtained and compared with the reference method.

### Data Analysis

Categorical agreement (CA) and Essential agreement (EA), when applicable, were calculated, as well as the errors quantified following the standard definition of ISO 20776-2 (18). Classification of the errors were performed as minor (mE), major (ME) and very major (VME). Proportion of agreement (PA), sensitivity and specificity for detecting ESBL in Enterobacterales group I and screening for pAmpC, carbapenemases and ESBL in Enterobacterales group II were also performed. Disk diffusion or micro-dilution (in the cases where disk diffusion assays were not recommended or there is a need to have a quantitative assay) were considered as gold standard methods and used for the comparison with the FASTinov kits results. Data analysis incorporated Expert Rules (EUCAST) for intrinsically resistant species to a certain drug on its software.

### (v) MALDI-TOF Identification

A total of 364 patient's positive blood cultures of which 177 were positive for gram-positive cocci and 187 were positive for gram-negative bacilli were processed according to instructions for use and protocols of FASTinov AST kits described above, and the resulting bacterial suspensions. However, for Maldi-TOF identification, the sample preparation step was conducted using Tergitol™ (Sigma) as follows.

A sterile microcentrifuge tube was filled with 1 ml of the blood culture and mixed with 50 μL of Tergitol (10% v/v), followed by vortex and centrifugation at 13000 rpm for 1 minute. The supernatant was discarded, and the resulting pellet was resuspended with 1 ml of sterile saline solution. The mixture was vortexed until the pellet was totally resuspended. 1 ml of this suspension was gently transferred on the top of a microcentrifuge tube filled with 500 μL of Histopaque®-1077. The centrifugation was repeated, then the supernatant was discarded, and the resulting pellet washed with saline solution, then centrifugated again and the supernatant discarded. The wash-centrifugation-discard cycle was repeated using sterile water and the final pellet was dried at 37° C. for 5-10 minutes.

The final pellet was used directly on the MALDI-TOF target plate in two spots for each sample (i.e., in duplicate), using a wooden toothpick and 1 μl of α-cyano-4-hydroxycinnamic acid (CHCA) matrix added after the spots dried. The target plate was placed in the Bruker MALDI Biotyper and the analysis was initiated, using the Sepsityper sample type option on the equipment. The results were compared with the colonies on the following day and the ID scores were recorded.

### (vi) Validation Study

### Study Design and Sample Collection.

The study was conducted in three sites simultaneously. These included:
First site: the FASTinov site in Porto (Portugal)

In this site, the study used BACTEC blood bottles from Becton Dickinson (BD) spiked with well-characterised strains belonging to the FASTinov bacteria collection, as well as ATCC strains from the American Type Culture Collection (ATCC) and incubated until positive result for infection was recorded, in line with the protocol described in reference 2.
Second site: the Hospital Ramon et Cajal, Madrid, a hospital with around 1000 beds.

Sequential patient's positive blood cultures (BACTEC, BD) were included in the study between November of 2021 to February 2022 (one sample per patient).
Third site: Centro Hospitalar S. João (CHSJ), Porto, a university hospital with around 1000 beds.

Sequential patient's positive blood cultures (BACTEC, BD) were included in the study (one sample per patient). The blood cultures were collected from patients with a suspected bloodstream infection between March of 2022 to July 2022.

The positive blood cultures were identified by matrix-assisted laser desorption ionization—time of flight (MALDI-TO F) mass spectrometry (Bruker Daltonics, Germany) using septityper mode. A great diversity of species was studied and reported on Table 12. All tested strains were sub-cultured on blood agar panels to assess purity, then reference susceptibility assays were performed on each strain and the strain was later frozen at −80° C. with the approved study codification. Polymicrobial blood cultures were excluded from the study.

### Ethical Considerations.

The study was approved by the ethical committee of the Ramón y Cajal University Hospital (reference no. 161/17) and by the ethical committee of Centro Hospitalar S. Joao (reference no. 284/21).

### FASTinov Assay.

Positive blood cultures already identified by Maldi-Tof from each site were processed according to the rapid AST of FASTinov kit instructions for use described above.

### Analysis of Cell Lesions

To evaluate cell lesions due to antibiotics, a flow cytometric analysis was performed using a Beckman Coulter CytoFlex model B3-R0-V3 (at site 1 and 2) and DxFlex (at site 3), both equipped with one blue laser (488 nm; output, 50 mW; beam spot size, 5 by 80 μm). The instruments have three fluorescence channels: 525/40 BP, 585/42 BP, and 690/50 BP. The DxFlex is equipped with a plate reader for automated analysis of each panel. The flow cytometers were used on slow mode.

### Software Analysis

A proprietary software with an algorithm defined by FASTinov was used for data analysis and the result considered on the comparison with a reference method.

### Timing of the Instrument

The time the panel spends inside the instrument is the bottleneck of the assay given that the equipment can only analyse one panel at the time. Therefore, the time required for the flow cytometry analysis of each panel only depends on the type of bacteria and the susceptibility protocols used (i.e., EUCAST or CLSI). This time is a fixed value assigned to the instrument given that it is programmed to analyse a fixed volume, acquisition in slow mode and automatically fixed washes between wells.

### Reproducibility

Ten inoculated samples used at the FASTinov site were also inoculated on each site in order to include a variety of phenotypes on each site and to calculate inter-laboratorial reproducibility.

### Reference Method (RM)

Positive blood cultures were spiked in blood agar and colonies identified and submitted to antimicrobial susceptibility assay according reference disk diffusion method and/or to MIC values determined by microdilution. The results were analysed using the most recent EUCAST breakpoint tables and the M100 (29th ed.) breakpoint tables from CLSI.

### Example 1—Optimization of the Sample Preparation Step

With regards to the sample preparation, FIGS. 1 and 2 show the number of CFUs of the cells recovered from aerobic (AR) and anaerobic (ANA) blood culture (BC) bottles using protocol A. For Gram negative bacteria, the final number of microorganisms (CFUs) obtained from AR and ANA bottles was not statistically different and always >superior to 1×10⁸/ml (>1×10⁸/ml).

Conversely, significant differences were observed for Staphylococcus spp, where the number of cells measured in ANA bottles was superior to the number measured in AR bottles for 8 out of 10 different strains grown in AR bottles. The number of Staphylococcus spp cells recovered was not enough to inoculate the FASTinov panels (which require a minimum of 1×10⁷/ml). Regarding Enterococcus spp., both AR and ANA bottles had enough cells to inoculate the FASTinov panels (>1×10⁷/ml), with slightly higher numbers measured in AR bottles. The data represented in Table 6 below corresponds to the mean of results obtained from 2 bottles per strain.

TABLE 6
Cell concentration obtained from positive cultures grown in both aerobic (AR)
and anaerobic (ANA) blood culture bottles after extraction with protocol A

| z | | | Staphylococcus spp. | | | Enterococcus spp. | | |
|---|---|---|---|---|---|---|---|---|
| | AR | ANA | | AR | ANA | | AR | ANA |
| IMP13476 | 6.28E+08 | 2.53E+08 | ATCC 25213 | 4.98E+05 | 1.04E+08 | ATCC 51299 | 4.79E+08 | 1.46E+08 |
| BAA1705 | 9.48E+08 | 4.00E+08 | ATCC 43300 | 7.60E+06 | 3.50E+07 | ATCC 29212 | 3.80E+08 | 1.60E+08 |
| NDM 13443 | 2.40E+08 | 1.24E+08 | ATCC 35984 | 3.70E+06 | 1.70E+07 | E018 | 1.88E+08 | 8.75E+07 |
| IMP8 | 2.99E+08 | 1.16E+08 | ST017 | 9.33E+06 | 2.50E+07 | E024 | 1.31E+08 | 1.69E+07 |
| ATCC 8739 | 4.47E+08 | 2.14E+08 | ST010 | 4.45E+06 | 1.86E+07 | E030 | 9.78E+07 | 7.95E+07 |
| ATCC 25922 | 3.60E+08 | 1.60E+08 | ST024 | 1.40E+07 | 3.86E+07 | E034 | 2.13E+08 | 5.64E+07 |
| ATCC 13048 | 2.55E+08 | 1.74E+08 | ST012 | 3.83E+06 | 1.84E+07 | E005 | 8.46E+07 | 3.03E+07 |
| CCUG 59627 | 2.62E+08 | 9.29E+08 | ST049 | 8.10E+06 | 6.31E+07 | E055 | 1.34E+08 | 5.55E+07 |
| ATCC 27853 | 1.15E+08 | 2.62E+08 | ATCC 25923 | 7.10E+06 | 5.53E+07 | E036 | 1.25E+08 | 2.10E+07 |
| ATCC 35218 | 6.82E+08 | 6.62E+08 | ST027 | 3.60E+07 | 1.55E+08 | E058 | 3.23E+08 | 4.90E+07 |
| Mean | 4.24E+08 | 3.29E+08 | Mean | 9.46E+06 | 5.30E+07 | Mean | 2.16E+08 | 7.02E+07 |
| p = 0.169 | | | p = 0.013 | | | p = 0.005 | | |

A—For Gram negative bacteria the final number of microorganisms measured from growth in AR and ANA bottles is not statistically different and always >1 × 10⁸/mL.
B1—Significant differences were observed for Gram positive Staphylococcus spp., where the number of cells measured in ANA bottles is superior to the number measured in AR bottles; for 8 out of 10 different strains grown in AR bottles, the number of cells measured was not enough to inoculate the FAST panel (minimum 1 × 10⁶/mL)
B2—For Gram positive Enterococcus spp., both AR and ANA bottles had a sufficient number of cells to inoculate the FAST panel (>1 × 10⁷/mL), with slightly higher numbers measured in AR bottles.
The data represented here correspond to the mean of results obtained from 2 bottles per each strain. Cell counting was performed by flow cytometry in a gate previously defined.

Comparing Protocols A and B, CFUs were higher on Protocol B in all cases (gram-negative bacilli and gram-positive cocci) as seen in Table 7 below. This difference was even more significant for Staphylococcus, given that enough cells to perform FASTinov assay could be obtained using Protocol B and AR BC bottles.

TABLE 7
Cell concentration obtained from positive cultures grown in both aerobic
(AR) and blood culture bottles after extraction with protocols A and B

| Gram-negative bacilli AR | | | Staphylococcus spp. AR | | | Enterococcus spp. AR | | |
|---|---|---|---|---|---|---|---|---|
| | Protocol A | Protocol B | | Protocol A | Protocol B | | Protocol A | Protocol B |
| IMP13476 | 6.28E+08 | 1.53E+09 | ATCC 25213 | 4.98E+05 | 1.40E+08 | ATCC 51299 | 4.79E+08 | 1.20E+09 |
| BAA1705 | 9.48E+08 | 2.50E+09 | ATCC 43300 | 7.60E+06 | 1.90E+08 | ATCC 29212 | 3.80E+08 | 1.30E+09 |
| NDM 13443 | 2.40E+08 | 9.80E+08 | ATCC 35984 | 3.70E+06 | 1.50E+08 | E018 | 1.88E+08 | 5.65E+08 |
| IMP8 | 2.99E+08 | 1.50E+09 | ST017 | 9.33E+06 | 1.70E+08 | E024 | 1.31E+08 | 2.10E+08 |
| ATCC 8739 | 4.47E+08 | 1.40E+09 | ST010 | 4.45E+06 | 1.90E+08 | E030 | 9.78E+07 | 1.50E+08 |
| ATCC 25922 | 3.60E+08 | 1.30E+09 | ST024 | 1.40E+07 | 2.60E+08 | E034 | 2.13E+08 | 2.50E+08 |
| ATCC 13048 | 2.55E+08 | 1.40E+09 | ST012 | 3.83E+06 | 1.40E+08 | E005 | 8.46E+07 | 2.30E+08 |
| CCUG 59627 | 2.62E+08 | 1.60E+09 | ST049 | 8.10E+06 | 2.60E+08 | E055 | 1.34E+08 | 1.40E+08 |
| ATCC 27853 | 1.15E+08 | 2.10E+09 | ATCC 25923 | 7.10E+06 | 5.90E+08 | E036 | 1.25E+08 | 2.40E+08 |
| ATCC 35218 | 6.82E+08 | 1.10E+09 | ST027 | 3.60E+07 | 1.60E+08 | E058 | 3.23E+08 | 1.80E+08 |
| Mean | 4.24E+08 | 1.54E+09 | Mean | 9.46E+06 | 2.25E+08 | Mean | 2.16E+08 | 4.47E+08 |
| p = 0.002 | | | p = 0.002 | | | p = 0.027 | | |

Protocol B recovered significantly more microorganisms than Protocol A; this is particularly relevant regarding Staphylococcus spp., inoculated on AR blood bottles.

In view of the results discussed above, and in order to simplify the method and provide a single sample preparation protocol that could work for all microorganisms and samples, Protocol B was selected for all of the cases moving forward. As shown in FIG. 1, protocol B involved the use of Triton X-100 and then, subsequently, Histopaque. The bacteria contained in the bacterial suspension obtained before preparing the subsequent suspension with a MacFarland optical concentration of 0.5, were correctly identified using MALDI-TOF Bruker.

Similar to what was observed with colonies from overnight blood cultures, the dried sample purified via Protocol B provided a high percentage of accurate identification of microorganisms: 100% on Gram-negative bacteria (Enterobacterales, Pseudomonas and Acinetobacter) as shown in FIG. 6A, and 98.3% on Gram positive cocci (Staphylococcus spp and Enterococcus spp) as shown in FIG. 6B.

This extraction protocol also gave excellent results with BioMérieux blood cultures bottles and the positive urines (with more than >10⁶ CFU/ml).

### Example 2—Selection of the Fluorescent Probes

The results of the susceptibility for each drug/antibiotic were compared with the reference method. Several combinations between drug/fluorescent probe gave good results. Table 8 below summarises the fluorescent probes tested in combination with the antibiotics disclosed in Tables 3 and 4.

TABLE 8
Fluorescent probes tested in combination with the antibiotics

| Fluorescent dye | Concentration | |
|---|---|---|
| CTC (5-Cyano-2,3-ditolyl tetrazolium chloride) | 4 | mM |
| CALCEIN AM | 2.5 | μg/mL |
| Dihydrorhodamine 123 | 2.5 to 10 μM | |
| DiBAC4(3) (Bis-(1,3-Dibutylbarbituric | 0.5/1/2 | μg/mL |
| Acid)Trimethine Oxonol) | | |
| DiOC2(3) (3,3′-Diethyloxacarbocyanine Iodide) | 0.03 | μM |
| Fluorescein Diacetate (FDA) | 0.5; 1 and 2 μM | |
| 5-CFDA, AM (5-Carboxyfluorescein Diacetate, | 0.5; 1 and 2 μM | |
| Acetoxymethyl Ester) | | |
| CFDA-SE (Carboxyfluorescein Diacetate | 0.5; 1 and 2 μM | |
| Succinimidyl Ester) | | |
| Propidium Iodide | 1 | μg/mL |
| SYTO ™ 16 Green Fluorescent Nucleic Acid Stain | 5, 10, 20 | μM |

CTC (5-Cyano-2,3-ditolyl tetrazolium chloride, Calceim AM, Dihydrorhodamine 123, DIBAC4(3), DioC 2(3), Fluorescein Diacetate, 5CFDA, AM, CFDA-SE, Propidium Iodine, SYTO 16 Green Fluorescent, Nucleic Acid Stain.

However, the number of selected fluorochromes was reduced to a minimum in order to reduce the number of controls, as each probe needs a positive and a negative control. In addition, the process of manufacturing development is more complex when the number of probes in the panel is increased. Furthermore, the inventors had to consider which probes will allow the panels to be stable at room temperature and the optimal drug/fluorochrome combinations. These processes are unique and complex and therefore needed extensive research and development.

For the FASTgramneg panel, which is used to test for Gram-negative bacteria, DiBAC₄(3) at 1 μg/ml was surprisingly identified as the probe to stain all the drugs except imipenem in case of Pseudomonas spp. Therefore, for this particular case, Propidium iodide (PI) at 1 μg/ml gave better results (see FASTgramneg panel layout in FIGS. 3A and 3B).

For the FASTgrampos panel, which is used to test for Gram-positive bacteria, two fluorescent probes: PI at 1 μg/ml and DiOC2(3) (3,3′-Diethyloxacarbocyanine Iodide) at 0.06 uM were selected according to the drugs of interest (see FASTgrampos panel layout in FIGS. 4A and 4B).

For the FASTcolistin MIC panel, which is used to test for Gram-negative bacteria PI at 1 μg/ml was optimum (see FASTcolistin MIC layout in FIGS. 5A and 5B). Non-treated cells were stained with those fluorescent probes (positive control) and the highest tested Benzydamine concentration tested was chosen for the negative control for all the probes.

### Example 3—Determination of the Bacterial Susceptibility Using the FASTinov Method and Associated Kits

Validation of all the processes together, which include the extraction protocol (protocol B) and the inoculation of the optimised panels, were all evaluated at the two sites and CA, EA and the number and type of errors found on the validation are shown in Table 9.

TABLE 9
Total performance data related to FASTgramneg, FASTgrampos, and

| | EUCAST | | | | | | | CLSI | | | | | | |
|---|---|---|---|---|---|---|---|---|---|---|---|---|---|---|
| Screening | N | EA % | CA % | mE | ME | VME | Reproducibility | N | EA % | CA % | mE | ME | VME | Reproducibility |
| FASTgramneg | 2570 | NA | 98.9 | 0.0% | 1.2% | 0.9% | 99.5% | 2677 | NA | 97.9 | 1.3% | 0.8% | 0.7% | 99.3% |
| FASTgrampos | 857 | 91.5 | 97.8 | 0.2% | 2.8% | 0.4% | 99.4% | 931 | 91.5 | 97.9 | 0.3% | 2.6% | 0.3% | 99.8% |
| FASTcolistin MIC | 254 | 94.9 | 98.8 | 0.0% | 0.0% | 1.2% | 97.0% | 254 | 94.9 | 98.8 | 0.0% | 0.0% | 1.2% | 97.0% |

n—number of strains
EA (%)—Percentage of Essential agreement
CA (%)—Percentage of Categorical Agreement
mE—minor Error;
ME—Major Error;
VME—Very Major Error

### FASTcolistin MIC

| | Detection of mechanisms of resistance | | | |
|---|---|---|---|---|
| | N | Sensitivity | Specificity | Accuracy |
| ESBL(EB group I) | 45 | 95.7% | 100% | 99.3% |
| ESBL(EB group II) | 17 | 100% | 100% | 100% |
| pAmpC | 37 | 100% | 100% | 100% |
| Carbapenemases | 52 | 92.2% | 95.1% | 94.1% |

### Example 4—MALDI-TOF Identification

The proportion of agreement (PA) of the FASTinov sample preparation method compared to the reference was around 95.6% overall. Detailed results are presented in Tables 10 and 11.

TABLE 10
Summary results of MALDI-TOF identification of samples prepared
with the FASTinov sample preparation method compared to the
Colony identification (reference) in Gram positive strains.

| | Colony IDs | FASTinov IDs |
|---|---|---|
| Gram-positive | (reference) | PA % |
| Staphylococcus spp. | 122 | 95.1 |
| S. aureus | 35 | 100 |
| S. capitis | 4 | 75.0 |
| S. epidermidis | 41 | 92.7 |
| S. haemolyticus | 6 | 83.3 |
| S. hominis | 35 | 97.1 |
| S. petrasii | 1 | 100 |
| Enterococcus spp. | 55 | 94.5 |
| E. faecalis | 39 | 92.3 |
| E. faecium | 15 | 100 |
| E. gallinarum | 1 | 100 |
| Total | 177 | |

TABLE 11
Summary results of MALDI-TOF identification of samples prepared
with the FASTinov sample preparation method compared to the
Colony identification (reference) in Gram negative strains.

| | Colony IDs | FASTinov IDs |
|---|---|---|
| Gram-negative | (reference) | PA % |
| Escherichia coli | 83 | 98.8 |
| K. oxytoca | 3 | 66.7 |
| K. pneumoniae | 50 | 92.0 |
| K. variicola | 1 | 100 |
| Proteus mirabilis | 11 | 100 |
| Morganella morganii | 1 | 100 |
| Serratia marcences | 5 | 100 |
| Enterobacter cloacae | 5 | 100 |
| Pantoea agglomerans | 1 | 100 |
| Pseudomonas aeruginosa | 21 | 95.2 |
| Acinetobacter baumannii | 5 | 100 |
| Acinetobacter variabilis | 1 | 100 |
| Total | 187 | |

As observed in the Tables, no misidentification was observed but in 16 cases out of a total 364, no identification was obtained; the scores were very similar to the colonies and divided by groups. The meaning of the score values was defined according to the Bruker MALDI Biotyper, using the cut-off values of Sepsityper sample type. The key findings from this study were as follows (higher scores correspond to better results):

In gram-positives: score <1.59 was observed in 14.3%;
scores between 1.6-1.79 in 4.1%
scores >1.8 in 81.6%.

In gram-negatives: the scores were higher, namely:
scores <1.59 was observed in 15.6%
score between 1.6-1.79 in none
scores >1.8 was observed in 84.4%

These results demonstrate that the FASTinov ultra-rapid AST sample preparation method can be used to run in parallel MALDI-TOF identification and ultra-rapid AST, for a highly time efficient diagnosis, while providing excellent accuracy for both gram-positive and gram-negative bacteria.

### Example 5—Validation Study

A total of 651 blood cultures were studied from the three sites, of which 348 isolates were gram-negative and 303 isolates were gram-positive. The distribution of the isolates by species per site is shown in Table 12.

TABLE 12
Distribution per site of the tested strains in the FASTgramneg
kit and FASTgrampos at FASTinov lab Porto (site 1),
Hospital Ramon y Cajal, Madrid (site 2) and Centro
Hospitalar S. João Porto (site 3).

| Gram-negative bacteria | Site 1 | Site 2 | Site 3 | Total |
|---|---|---|---|---|
| Enterobacterales | 81 | 76 | 76 | 233 |
| E. coli | 15 | 49 | 38 | 102 |
| Klebsiella pneumoniae | 41 | 13 | 25 | 79 |
| Kl. aerogenes | 4 | 1 | 2 | 7 |
| Kl. oxytova | — | 2 | 1 | 3 |
| Kl. variicola | — | — | 1 | 1 |
| E. cloacae | 9 | — | — | 9 |
| E. kobei | — | 1 | — | 1 |
| Citrobacter koseri | — | 2 | — | 2 |
| C. freundii | — | 1 | — | 1 |
| Proteus mirabilis | 5 | 4 | 4 | 13 |
| Serratia marcencens | 3 | — | 2 | 5 |
| S. nematodiphila | — | — | 1 | 1 |
| Morganella morgannii | — | — | 2 | 2 |
| Samonella enteritidis | 2 | — | — | 2 |
| S. thyphimurium | 1 | — | — | 1 |
| Providencia rettegeri | 1 | — | — | 1 |
| Non-fermentors | 102 | 7 | 6 | 115 |
| Pseudomonas aeruginosa | 72 | 5 | 5 | 82 |
| Acinetobacter baummanii | 30 | — | — | 33 |
| A. calcoaceticus | — | 1 | — | 2 |
| A. pitti | — | 1 | — | 1 |
| A. variabilis | — | — | 1 | 1 |
| N total | 183 | 83 | 82 | 348 |
| Gram-positive bacteria | Site 1 | Site 2 | Site 3 | Total |
| Staphylococcus spp | 66 | 46 | 92 | 204 |
| S. aureus | 35 | 12 | 16 | 63 |
| S. epidermidis | 23 | 24 | 41 | 88 |
| S. capitis | — | 1 | 12 | 13 |
| S. hominis | 8 | 7 | 17 | 32 |
| S. haemolyticus | — | 1 | 3 | 4 |
| S. simulans | — | — | 1 | 1 |
| S. lugdunensis | — | 1 | 1 | 2 |
| S. warneri | — | — | 1 | 1 |
| Enterococcus spp | 65 | 22 | 12 | 99 |
| E. faecalis | 44 | 10 | 7 | 61 |
| E. faecium | 18 | 9 | 5 | 32 |
| E. gallinarum | 2 | 2 | — | 4 |
| E. casseiflavus | 1 | — | — | 1 |
| E. raffinosus | — | 1 | — | 1 |
| N total | 131 | 68 | 104 | 303 |

Based on the analysis of the results obtained from the multi-sites study with the EUCAST/CLSI guidelines, the sensitivity and specificity of the test was superior to 90% as shown on Table 13. Reproducibility with the FASTgramneg panel was 96.8%/95% and for the FASTgrampos panel was 95.1%/95.1% according EUCAST/CLSI.

TABLE 13
Sensitivity and specificity according both EUCAST and CLSI protocols
of the FASTinov kits when compared to reference method.

| | EUCAST/CLSI | Site 1 | Site 2 | Site 3 | Total |
|---|---|---|---|---|---|
| FASTgramneg | Sensitivity (%) | 98.8/99.2 | 97.1/96.6 | 98.5/100 | 98.5/98.9 |
| | Specificity (%) | 98.2/96.5 | 99.3/97.5 | 99.5/100 | 99.0/98.9 |
| FASTgrampos | Sensitivity (%) | 100/100 | 98.5/99.0 | 100/100 | 99.8/99.8 |
| | Specificity (%) | 96.9/96.8 | 97.5/97.6 | 97.5/97.5 | 97.3/97.2 |

Time needed to read the FASTinov panel by the flow cytometer's software bioFAST was dependent on the number of drugs and concentrations tested for each microorganism and the selected protocol. Recorded time values are reported in Table 14 below. The minimum time was 9 min for Acinetobacter spp on EUCAST protocol and the highest, 47 min with Enterobacterales according CLSI.

TABLE 14
Time required to read the FASTinov panel by the
flow cytometer; according to bioFAST software

| Type of bacteria | EUCAST | CLSI |
|---|---|---|
| Enterobacterales | 39 min | 47 min |
| Pseudomonas spp | 15 min | 27 min |
| Acinetobacter spp | 9 min | 23 min |
| Staphylococcus spp | 27 min | 29 min |
| Enterococcus spp | 20 min | 20 min |

### Very Major Results (VME)

Details of the VME are presented in Table 15. A higher number of strains was observed with piperacillin/tazobactam and ceftalozane/tazobactam strains (4 strains each). Regarding amoxacillin/clavulanic acid (EUCAST) and ceftazidime/avibactam and amikacin (both on EUCAST/CLSI) the CA was 100%.

TABLE 15
Very Major Results (VME) obtained with FASTgramneg and FASTgrampos
kits according to EUCAST and/or CLSI protocols per site (site
1-FASTinov, Porto; site 2- Hospital Ramon et Cajal, Madrid;
site 3- Centro Hospitalar S. João, Porto)

| | | | | MIC | | |
|---|---|---|---|---|---|---|
| Drug | n | Strain | Identification | (mg/L) | Protocol | Site |
| Ampicillin | 1 | EB 106 | E. coli | 16 | EUCAST | 2 |
| Cefepime | 3 | EB 022 | E. coli | 4 | EUCAST | 2 |
| | | EB 033 | K. pneumoniae | 32 | EUCAST/CLSI | 2 |
| | | EB 052 | E. coli | 64 | CLSI | 2 |
| Ceftazidime | 3 | EB 1168 | E. cloacae | 32 | EUCAST/CLSI | 1 |
| | | EB 033 | k. pneumoniae | 32 | CLSI | 2 |
| | | EB 069 | K. aerogenes | 16 | CLSI | 2 |
| Piperacillin/ | 4 | EB 101 | k. pneumoniae | 64 | EUCAST | 1 |
| tazobactam | | EB 123 | E. cloacae | 64/4 | EUCAST | 1 |
| | | EB 718 | E. coli | >128/4 | EUCAST/CLSI | 1 |
| | | EB 069 | K. aerogenes | 64/4 | EUCAST | 2 |
| Ceftalozane/ | 4 | EB 101 | K. pneumoniae | >32 | EUCAST/CLSI | 1 |
| tazobactam | | EB 677 | K. pneumoniae | 16 | EUCAST/CLSI | 1 |
| | | EB 679 | K. pneumoniae | >32 | EUCAST/CLSI | 1 |
| | | EB 032 | K. pneumoniae | 4 | EUCAST | 3 |
| Meropenem | 1 | EB 021 | K. pneumoniae | 16 | EUCAST/CLSI | 2 |
| Gentamicin | 1 | EB 286 | E. coli | 8 | EUCAST | 3 |
| Penicillin | 1 | ST 099 | S. aureus | 0.25 | EUCAST/CLSI | 2 |

### Results Observed on the FASTgramneq Kit

The overall categorical agreement (CA) of the FASTgramneg kit was >95% with errors of <1.5%.

53 of Enterobacterales group I were ESBL positive on reference method (RM) and FASTinov assay had sensitivity and specificity of 96.2% and 100%, respectively, with a PA of 99.0%. Regarding the screening for ESBL on Enterobacterales of group II, 13 were positive being the sensitivity, specificity was 100%, with a PA of 100%. For plasmid AmpC screening (Enterobacterales group 1), 38 were positive on RM being the sensitivity and specificity as well as the proportion of agreement (PA) 100%. 52 of the strains were positive for carbapenemases (MIC>0.25 ug/ml for meropenem) and FASTinov test showed 96.2% sensitivity (2 false negative were found with strains with MIC of 0.5 ug/ml) and 96.7% specificity with a PA of 96.6%.

TABLE 16
FASTgramneg results obtained with the total of 3 sites compared with reference method

| | FASTgramneg: total on blood cultures | | | | | | | | | | | | | | | | |
|---|---|---|---|---|---|---|---|---|---|---|---|---|---|---|---|---|---|
| | EUCAST | | | | | | | | CLSI | | | | | | | | |
| Antimicrobial | RM | | | | | | | | RM | | | | | | | | |
| agent | n | S | I | R | CA(%) | mE | ME | VME | n | S | I | SDD | R | CA(%) | mE | ME | VME |
| Ampicillin | 231 | 66 | — | 165 | 99.6 | — | — | 1/165 | 231 | 66 | 1 | — | 164 | 99.1 | 2/231 | — | — |
| Amoxacillin- | 231 | 106 | — | 125 | 100 | — | — | — | 231 | 110 | 14 | — | 107 | 96.9 | 7/231 | — | — |
| clavulanic acid | | | | | | | | | | | | | | | | | |
| Cefotaxime | 231 | 156 | — | 75 | 98.7 | — | 3/156 | — | 231 | 156 | — | — | 75 | 98.3 | 2/231 | 2/156 | — |
| Ceftazidime | 312 | 159 | 34 | 119 | 98.7 | 1/312 | 2/159 | 1/119 | 313 | 194 | 4 | — | 115 | 97.1 | 5/313 | 1/194 | 3/115 |
| Cefepime | 313 | 165 | 44 | 104 | 98.7 | 1/313 | 1/165 | 2/104 | 314 | 210 | 4 | 5 | 95 | 97.5 | 6/314 | — | 2/95 |
| Piperacillin- | 314 | 177 | 51 | 86 | 97.1 | — | 5/177 | 4/86 | 345 | 237 | 13 | — | 95 | 94.5 | 13/345 | 5/237 | 1/95 |
| tazobactam | | | | | | | | | | | | | | | | | |
| Ceftolozane- | 312 | 267 | — | 45 | 97.4 | — | 4/267 | 4/45 | 312 | 267 | 5 | — | 40 | 96.2 | 6/312 | 3/267 | 3/40 |
| tazobactam | | | | | | | | | | | | | | | | | |
| Ceftazidime- | 315 | 307 | — | 8 | 100 | — | — | — | 315 | 307 | — | — | 8 | 100 | — | — | — |
| avibactam | | | | | | | | | | | | | | | | | |
| Meropenem | 232 | 223 | — | 9 | 99.6 | — | — | 1/9 | 232 | 217 | 5 | — | 10 | 98.7 | 2/232 | — | 1/10 |
| Ciprofloxacin | 347 | 159 | 55 | 133 | 98.8 | 2/347 | 2/159 | | 347 | 213 | 6 | — | 128 | 98.8 | 3/347 | 1/213 | — |
| Gentamicin | 263 | 194 | — | 69 | 99.2 | — | 1/194 | 1/69 | 345 | 262 | 6 | — | 77 | 99.4 | 2/345 | — | — |
| Amikacin | 343 | 313 | — | 30 | 100 | — | — | — | 343 | 316 | — | — | 27 | 100 | — | — | — |
| Overall | 3444 | 2292 | 184 | 968 | 98.9 | 0.1 | 0.8 | 1.4 | 3559 | 2555 | 58 | 5 | 941 | 98.0 | 1.3 | 0.5 | 1.1 |

### FASTgrampos Kit

The Gram-positive kits analysed by EUCAST guidelines achieved a sensitivity and specificity >95% and a CA >95% (Table 17). All tested drugs showed a CA of >90%. The ME and mE were also low.

TABLE 17
FASTgrampos results obtained with the total of 3 sites compared with reference method

| | FASTgrampos total of blood cultures | | | | | | | | | | | | | | | | | |
|---|---|---|---|---|---|---|---|---|---|---|---|---|---|---|---|---|---|---|
| Antimi- | EUCAST | | | | | | | | | CLSI | | | | | | | | |
| crobial | RM | | | | | | | | | RM | | | | | | | | |
| agent | n | S | I | R | EA(%) | CA(%) | mE | ME | VME | n | S | I | R | EA(%) | CA(%) | mE | ME | VME |
| Penicillin* | 75 | 19 | — | 56 | — | 94.7 | — | 3/19 | 1/56 | 303 | 94 | — | 209 | — | 98.3 | — | 4/94 | 1/209 |
| Ampicillin | 99 | 59 | 2 | 38 | — | 98.0 | 2/99 | — | — | 99 | 61 | — | 38 | — | 98.9 | — | 1/61 | — |
| Cefox- | 100 | 48 | — | 52 | — | 96.0 | — | 4/48 | — | 100 | 48 | — | 52 | — | 96.0 | — | 4/48 | — |
| itin** | | | | | | | | | | | | | | | | | | |
| Oxacil- | 88 | 14 | — | 74 | — | 95.5 | — | 4/14 | — | 88 | 14 | — | 74 | — | 95.5 | — | 4/14 | — |
| lin** | | | | | | | | | | | | | | | | | | |
| Vanco- | 265 | 254 | — | 11 | 92.1 | 98.9 | — | 3/254 | — | 265 | 254 | 3 | 8 | 92.1 | 98.9 | — | 3/254 | — |
| mycin | | | | | | | | | | | | | | | | | | |
| Linezolid | 303 | 298 | — | 5 | — | 98.3 | 2/303 | 3/298 | — | 303 | 298 | — | 5 | — | 98.0 | 3/303 | 3/298 | — |
| Gentami- | 201 | 131 | — | 70 | — | 99.0 | — | 2/131 | — | 201 | 147 | — | 54 | — | 98.5 | 1/201 | 2/147 | — |
| cin | | | | | | | | | | | | | | | | | | |
| Gentami- | 66 | 51 | — | 15 | — | 95.5 | — | 3/51 | — | 66 | 51 | — | 15 | — | 96.9 | — | 2/51 | — |
| cin high | | | | | | | | | | | | | | | | | | |
| level | | | | | | | | | | | | | | | | | | |
| Overall | 1197 | 874 | 2 | 321 | | 97.7 | 0.3% | 2.5% | 0.3% | 1425 | 967 | 3 | 455 | | 0.3% | 98.0 | 2.4% | 0.2% |

Penicillin*—only for S. aureus on EUCAST
Cefoxitin**—except S. epidermidis
Oxacillin***—only S. epidermidis

The Essential agreement (EA) for MIC determination to vancomycin on S. aureus was 100%, with a bias of −30%, which is on the inferior limit recommended on ISO 20776-2: 2021.

Table 18 shows the distribution of MIC of the S. aureus tested. The FASTinov method often provides one dilution above the standard method.

TABLE 18
Distribution of MIC values of Staphylococcus aureus to vancomycin
obtained on the FASTgrampos kit and broth microdilution (reference
method), essential agreement (EA %) and bias calculation.

MIC by microdilution

| | mg/L | 0.125 | 0.25 | 0.5 | 1 | 2 |
|---|---|---|---|---|---|---|
| MIC by | 0.125 | | | | | |
| FASTgrampos | 0.25 | | 2 | 15 | | |
| | 0.5 | | | 21 | 10 | |
| | 1 | | | 3 | 7 | |
| | 2 | | | | 3 | 2 |

The results obtained in each individual sites are provided in Tables 19-24 below.

TABLE 19
Total Fastgramneg performed at FASTionv, Porto

| | FASTgramneg: total spiked on blood cultures | | | | | | | | | | | | | | | | |
|---|---|---|---|---|---|---|---|---|---|---|---|---|---|---|---|---|---|
| | EUCAST | | | | | | | | CLSI | | | | | | | | |
| Antimicrobial | RM | | | | | | | | RM | | | | | | | | |
| agent | n | S | I | R | CA(%) | mE | ME | VME | n | S | I | SDD | R | CA(%) | mE | ME | VME |
| Ampicillin | 81 | 14 | — | 67 | 100 | — | — | — | 81 | 14 | — | — | 67 | 100 | — | — | — |
| Amoxacillin- | 81 | 19 | — | 62 | 100 | — | — | — | 81 | 20 | 5 | — | 56 | 96.3 | 3/81 | — | — |
| clavulanic acid | | | | | | | | | | | | | | | | | |
| Cefotaxime | 81 | 27 | — | 54 | 97.5 | — | 2/27 | — | 81 | 27 | — | — | 54 | 97.5 | — | 2/27 | — |
| Ceftazidime | 153 | 26 | 29 | 98 | 98.0 | — | 2/26 | 1/98 | 153 | 56 | 3 | — | 94 | 98.7 | — | 1/56 | 1/94 |
| Cefepime | 153 | 32 | 39 | 82 | 99.3 | — | 1/32 | — | 153 | 72 | 1 | 2 | 78 | 99.3 | 1/153 | — | — |
| Piperacillin- | 153 | 33 | 49 | 71 | 94.8 | — | 5/33 | 3/71 | 183 | 90 | 6 | — | 87 | 92.9 | 7/153 | 5/90 | 1/87 |
| tazobactam | | | | | | | | | | | | | | | | | |
| Ceftolozane- | 153 | 112 | — | 41 | 95.4 | — | 4/112 | 3/41 | 153 | 112 | 4 | — | 37 | 92.8 | 5/153 | 3/112 | 3/37 |
| tazobactam | | | | | | | | | | | | | | | | | |
| Ceftazidime- | 153 | 145 | — | 8 | 100 | — | — | — | 153 | 145 | — | — | 8 | 100 | — | — | — |
| avibactam | | | | | | | | | | | | | | | | | |
| Meropenem | 81 | 75 | — | 6 | 100 | — | — | — | 81 | 69 | 5 | — | 7 | 97.5 | 2/81 | — | — |
| Ciprofloxacin | 183 | 39 | 48 | 96 | 99.5 | 1/183 | | — | 183 | 87 | 3 | — | 93 | 99.5 | 1/183 | — | — |
| Gentamicin | 111 | 64 | — | 47 | 100 | — | — | — | 183 | 120 | 3 | — | 60 | 99.5 | 1/183 | — | — |
| Amikacin | 183 | 156 | — | 27 | 100 | — | — | — | 183 | 158 | — | — | 25 | 100 | — | — | — |
| Overall | 1566 | 742 | 165 | 659 | 98.6 | 0.06% | 1.9% | 1.1% | 1668 | 970 | 30 | 2 | 666 | 97.8 | 1.2% | 1.1% | 0.8% |

TABLE 20
Total Fastgrampos performed at FASTionv, Porto

| | FASTgrampos total spiked on blood cultures | | | | | | | | | |
|---|---|---|---|---|---|---|---|---|---|---|
| | EUCAST | | | | | | | | | CLSI |
| Antimicrobial | RM | | | | | | | | | RM |
| agent | n | S | I | R | EA(%) | CA(%) | mE | ME | VME | n |
| Penicillin* | 35 | 7 | — | 28 | — | 94.3 | — | 2/7 | — | 131 |
| Ampicillin | 65 | 42 | — | 23 | — | 98.5 | 1/65 | — | — | 65 |
| Cefoxitin** | 43 | 26 | — | 17 | — | 95.3 | — | 2/26 | — | 43 |
| Oxacillin***—only | 23 | 4 | — | 19 | — | 91.3 | — | 2/4 | — | 23 |
| S. epidermidis | | | | | | | | | | |
| Imipenem | 65 | — | 39 | 26 | — | 100 | — | — | — | NA |
| Vancomycin | 110 | 100 | — | 10 | 100 | 98.2 | — | 2/100 | — | 110 |
| Linezolid | 131 | 128 | — | 3 | — | 99.2 | — | 1/128 | — | 131 |
| Gentamicin | 66 | 37 | — | 29 | — | 98.5 | — | 1/37 | — | 66 |
| Gentamicin | 47 | 35 | — | 12 | — | 95.5 | — | 2/35 | — | 47 |
| high level | | | | | | | | | | |
| Overall | 585 | 379 | 39 | 167 | 100 | 97.8 | 0.1% | 3.2% | 0.0% | 616 |

| | FASTgrampos total spiked on blood cultures | | | | | | | |
|---|---|---|---|---|---|---|---|---|
| | CLSI | | | | | | | |
| Antimicrobial | RM | | | | | | | |
| agent | S | I | R | EA(%) | CA(%) | mE | ME | VME |
| Penicillin* | 49 | — | 82 | — | 97.7 | — | 3/49 | — |
| Ampicillin | 42 | — | 23 | — | 100 | — | — | — |
| Cefoxitin** | 26 | — | 17 | — | 95.3 | — | 2/26 | — |
| Oxacillin***—only | 4 | — | 19 | — | 91.3 | — | 2/4 | — |
| S. epidermidis | | | | | | | | |
| Imipenem | NA | NA | NA | — | — | — | — | — |
| Vancomycin | 100 | 3 | 7 | 100 | 98.2 | — | 2/100 | — |
| Linezolid | 128 | — | 3 | — | 98.5 | 1/131 | 1/128 | — |
| Gentamicin | 51 | — | 15 | — | 97.0 | 1/66 | 1/51 | — |
| Gentamicin | 35 | — | 12 | — | 97.9 | — | 1/35 | — |
| high level | | | | | | | | |
| Overall | 435 | 3 | 178 | 100 | 97.7 | 0.3% | 2.8% | 0.0% |

Penicillin*—only for S. aureus on EUCAST
Cefoxitin**—except S. epidermidis
Oxacillin***—only S. epidermidis

TABLE 21
Total Fastgramneg performed at Hospital Ramon et Cajal, Madrid

| | FASTgramneg Total of patients'blood cultures | | | | | | | | | | | | | | | | |
|---|---|---|---|---|---|---|---|---|---|---|---|---|---|---|---|---|---|
| | EUCAST | | | | | | | | CLSI | | | | | | | | |
| | RM | | | | | | | | RM | | | | | | | | |
| Antimicrobial agent | n | S | I | R | CA(%) | mE | ME | VME | n | S | I | SDD | R | CA(%) | mE | ME | VME |
| Ampicillin | 74 | 20 | — | 54 | 98.6 | — | — | 1/54 | 74 | 20 | 1 | — | 53 | 97.3 | 2/74 | — | — |
| Amoxacillin-clavulanic | 74 | 44 | — | 30 | 100 | — | — | — | 74 | 46 | 9 | — | 19 | 94.6 | 4/74 | — | — |
| acid | | | | | | | | | | | | | | | | | |
| Cefotaxime | 74 | 61 | — | 13 | 98.7 | — | 1/61 | — | 74 | 61 | — | — | 13 | 96.0 | 2/74 | — | — |
| Ceftazidime | 78 | 60 | 3 | 15 | 100 | — | — | — | 78 | 64 | 1 | — | 13 | 92.3 | 4/78 | — | 2/13 |
| Cefepime | 79 | 62 | 4 | 13 | 97.5 | — | — | 2/13 | 79 | 66 | — | 3 | 10 | 93.7 | 3/79 | — | 2/10 |
| Piperacillin- | 80 | 69 | 2 | 9 | 98.8 | — | — | 1/9 | 80 | 71 | 7 | — | 2 | 96.3 | 3/80 | — | — |
| tazobactam | | | | | | | | | | | | | | | | | |
| Ceftolozane- | 78 | 75 | — | 3 | 100 | — | — | — | 78 | 75 | — | — | 3 | 100 | — | — | — |
| tazobactam | | | | | | | | | | | | | | | | | |
| Ceftazidime-avibactam | 81 | 81 | — | — | 100 | — | — | — | 81 | 81 | — | — | 1 | 100 | — | — | — |
| Meropenem | 75 | 72 | — | 3 | 98.7 | — | | 1/3 | 75 | 72 | — | — | 3 | 98.7 | — | | 1/3 |
| Ciprofloxacin | 82 | 53 | 7 | 22 | 98.8 | 1/82 | — | — | 82 | 59 | 3 | — | 20 | 98.8 | 1/82 | — | — |
| Gentamicin | 75 | 65 | — | 10 | 97.3 | — | 2/65 | — | 80 | 70 | 1 | — | 9 | 100 | — | — | — |
| Amikacin | 78 | 77 | — | 1 | 100 | — | — | — | 78 | 77 | — | — | 1 | 100 | — | — | — |
| Overall | 928 | 739 | 16 | 173 | 99.0 | 0.1% | 0.4% | 2.9% | 933 | 762 | 22 | 3 | 146 | 97.3 | 2.0% | — | 3.4% |

TABLE 22
Total Fastgrampos performed at Hospital Ramon et Cajal, Madrid

| | FASTgrampos Total patients blood cultures | | | | | | | | | | | | | | | |
|---|---|---|---|---|---|---|---|---|---|---|---|---|---|---|---|---|
| | EUCAST | | | | | | | | CLSI | | | | | | | |
| Antimicrobial | RM | | | | | | | | RM | | | | | | | |
| agent | n | S | I | R | CA(%) | mE | ME | VME | n | S | SI | R | CA(%) | mE | ME | VME |
| Penicillin* | 12 | 2 | — | 10 | 91.7 | — | — | 1/10 | 68 | 15 | — | 53 | 98.5 | — | — | 1/53 |
| Ampicillin | 22 | 10 | 2 | 10 | 95.5 | 1/22 | — | — | 22 | 12 | — | 10 | 95.5 | — | 1/12 | — |
| Cefoxitin** | 22 | 13 | — | 9 | 95.5 | — | 1/13 | — | 22 | 13 | — | 9 | 95.5 | — | 1/13 | — |
| Oxacillin** | 24 | 5 | — | 19 | 100 | — | — | — | 24 | 5 | — | 19 | 100 | — | — | — |
| Imipenem | 13 | — | 12 | 1 | 100 | — | — | — | NA | NA | NA | NA | — | — | — | — |
| Vancomycin | 56 | 56 | — | — | 100 | — | — | — | 56 | 56 | — | — | 100 | — | — | — |
| Linezolid | 68 | 68 | — | — | 97.1 | — | 2/68 | — | 68 | 68 | — | — | 97.1 | — | 2/68 | — |
| Gentamicin | 43 | 26 | — | 17 | 97.7 | — | 1/26 | — | 43 | 28 | — | 15 | 97.7 | — | 1/28 | — |
| Gentamicin high level | 12 | 10 | — | 2 | 100 | — | — | — | 12 | 10 | — | 2 | 100 | — | — | — |
| Overall | 272 | 190 | 14 | 68 | 97.8 | 0.4% | 2.1% | 1.5% | 315 | 207 | — | 108 | 98.1 | 0.0% | 2.4% | 0.9% |

Penicillin*—only for S. aureus on EUCAST
Cefoxitin**—except S. epidermidis
Oxacillin***—only S. epidermidis

TABLE 23
Total Fastgramneg performed at Hospital S. João, Porto

| | FASTgramneg Total of ‘patients’blood cultures | | | | | | | | | | | | | | | |
|---|---|---|---|---|---|---|---|---|---|---|---|---|---|---|---|---|
| | EUCAST | | | | | | | | CLSI | | | | | | | |
| | RM | | | | | | | | RM | | | | | | | |
| Antimicrobial agent | n | S | I | R | CA(%) | mE | ME | VME | n | S | I | R | CA(%) | mE | ME | VME |
| Ampicillin | 76 | 32 | — | 44 | 100 | — | — | — | 76 | 32 | — | 44 | 100 | — | — | — |
| Amoxacillin-clavulanic acid | 76 | 43 | — | 33 | 100 | — | | — | 76 | 44 | — | 32 | 100 | — | — | — |
| Cefotaxime | 76 | 68 | — | 8 | 100 | — | — | — | 76 | 68 | — | 8 | 100 | — | — | — |
| Ceftazidime | 81 | 73 | 2 | 6 | 98.8 | 1/81 | — | — | 82 | 74 | — | 8 | 98.8 | 1/82 | — | — |
| Cefepime | 81 | 71 | 1 | 9 | 98.8 | 1/81 | — | — | 82 | 72 | 3 | 7 | 97.6 | 2/82 | — | — |
| Piperacillin-tazobactam | 81 | 75 | — | 6 | 100 | — | — | — | 82 | 76 | — | 6 | 96.3 | 3/82 | — | — |
| Ceftolozane-tazobactam | 81 | 80 | — | 1 | 98.7 | — | — | 1/1 | 81 | 80 | 1 | — | 98.8 | 1/81 | — | — |
| Ceftazidime-avibactam | 81 | 81 | — | — | 100 | — | — | — | 81 | 81 | — | — | 100 | — | — | — |
| Meropenem | 76 | 76 | — | — | 100 | — | — | — | 76 | 76 | — | — | 100 | — | — | — |
| Ciprofloxacin | 82 | 67 | — | 15 | 97.6 | — | 2/67 | — | 82 | 67 | — | 15 | 97.6 | 1/82 | 1/67 | — |
| Gentamicin | 77 | 65 | — | 12 | 98.7 | — | — | 1/12 | 82 | 72 | 2 | 8 | 98.8 | 1/82 | — | — |
| Amikacin | 82 | 80 | — | 2 | 100 | — | — | — | 82 | 81 | — | 1 | 100 | — | — | — |
| Overall | 950 | 811 | 3 | 136 | 99.4 | 0.2% | 0.2% | 1.5% | 958 | 823 | 6 | 129 | 99.1 | 0.9% | 0.1% | — |

TABLE 24
Total Fastgrampos performed at Hospital S. João, Porto

| | FASTgrampos total patients' blood cultures | | | | | | | | | | | | | | | | | |
|---|---|---|---|---|---|---|---|---|---|---|---|---|---|---|---|---|---|---|
| | EUCAST | | | | | | | | | CLSI | | | | | | | | |
| Antimicrobial | RM | | | | | | | | | RM | | | | | | | | |
| agent | n | S | I | R | EA(%) | CA(%) | mE | ME | VME | n | S | I | R | EA(%) | CA(%) | mE | ME | VME |
| Penicillin* | 28 | 10 | — | 18 | — | 96.4 | — | 1/10 | — | 104 | 30 | — | 74 | | 99.0 | — | 1/30 | — |
| Ampicillin | 12 | 7 | — | 5 | — | 100 | — | — | — | 12 | 7 | — | 5 | | 100 | — | — | — |
| Cefoxitin** | 35 | 9 | — | 26 | — | 97.1 | — | 1/9 | — | 35 | 9 | — | 26 | | 97.1 | — | 1/9 | — |
| Oxacillin** | 41 | 5 | — | 36 | — | 95.1 | — | 2/5 | — | 41 | 5 | — | 36 | | 95.1 | — | 2/5 | — |
| Vancomycin | 99 | 98 | — | 1 | 93.8 | 99.0 | — | 1/98 | — | 99 | 98 | — | 1 | 93.8 | 98.9 | — | 1/98 | — |
| Linezolid | 104 | 102 | — | 2 | — | 98.1 | 2/104 | — | — | 104 | 102 | — | 2 | | 98.1 | 2/104 | — | — |
| Gentamicin | 92 | 68 | — | 24 | — | 100 | — | — | — | 92 | 68 | — | 24 | | 100 | — | — | — |
| Gentamicin | 7 | 6 | — | 1 | — | 85.7 | — | 1/6 | — | 7 | 6 | — | 1 | | 85.7 | — | 1/6 | — |
| high level | | | | | | | | | | | | | | | | | | |
| Overall | 418 | 305 | — | 113 | — | 98.1 | 0.5% | 1.9% | — | 494 | 325 | — | 169 | | 98.1 | 0.4% | 1.8% | — |

Penicillin*—only for S. aureus on EUCAST
Cefoxitin**—except S. epidermidis
Oxacillin***—only S. epidermidis

### Discussion and Conclusions

Rapid antimicrobial assays are urgently needed in hospitals, especially regarding critical situations, such as sepsis. The inventors have developed a highly innovative and disruptive technology of Antimicrobial Susceptibility Tests (AST) evaluation together with an improved protocol for efficiently extracting microorganisms from clinical samples in concentrations that allow them to be readily analysed by flow cytometry. A clean (with as little debris as possible) suspension of microorganisms, at a concentration of at least 1×10⁷/ml, was needed to perform the novel and rapid AST assay. The inventors started the extraction of microorganisms (bacteria or fungi) from a positive blood culture or urine culture, or any other biological sample, using a barricot tube (Protocol A described above). In the process, some of the bacterial cells were lost during centrifugation (the bacterial cells formed a pellet together with human/animal cells and debris at the bottom of the tube) although, in most of the cases, a suspension with enough cells could be obtained. The most problematic cases were observed with Staphylococcus on aerobic blood cultures, likely due to its usual conformation in liquid media: grape-like cluster would go to the bottom with centrifugation. This situation, however, did not happen on anaerobic blood cultures, possibly because they include saponin, which enables the formation of big grape-like clusters during the division process.

Knowing that Staphylococcus is one of the most common microorganisms recovered from blood cultures in clinical settings and that anaerobic blood bottles are the most common blood cultures in the labs, the inventors have developed an improved extraction process, i.e. protocol B described above and as shown in FIG. 1, that has proven to be high effective for the extraction of micro-organisms, and Staphylococcus in particular.

Histopaque®-1077 can make a concentration gradient of the blood components and is especially used to separate white blood cells (on the top of Histopaque®-1077) from red blood cells (on the bottom). When the blood sample include microorganisms, they will go to the bottom together with the red blood cells. Using a lysed blood culture sample (treated with lysing agent like Triton X-100 or Tergitol) before adding the suspension to the Histopaque®-1077, clean and concentrated suspensions of microorganisms were obtained after centrifugation with Histopaque®-1077. Protocol B was especially useful when the microorganism that needed to be extracted is Staphylococcus. However, it can be used when trying to extract any and all microorganism species, both with aerobic or anaerobic blood cultures in bottles (BD or BioMérieux), and, surprisingly, even with other biological samples, such as urine, bronchial secretions, and cerebrospinal fluid. This is therefore the first report of the novel extraction protocol developed by the inventors.

Concerning the panels (i.e. kits) developed by the inventors, several fluorescent probes could be used for each antimicrobial agent. However, the inventors decided to use the same probes for most of the cases when possible, because each probe that is used needs a corresponding positive control and a negative control. Therefore, if several probes were included, this would negatively affect and prolong the time needed to perform the assay and to generate the antibiotic susceptibility reading. Furthermore, the process of drying each probe is complex and, therefore, the inventors chose to include as few probes as possible.

Three controls were designed for this assay. Firstly, the positive control, which is a classically used control in microbiology assays to assure the viability of the strain. The two other controls, namely the auto-fluorescence and negative controls are typically used for cytometry assays, to confirm whether the stain is present (i.e., a difference between stained and non-stained cells) and that the probe properly stains the cells. This is the first report of their use in a microbiology assay.

In summary, therefore, the assay comprises a positive control to ascertain bacterial viability. The positive control is achieved with non-treated bacterial cells with antibiotics, but exposed to fluorescent staining that were confirmed during development as not affecting the bacterial cell viability. In the Autofluorescence control, the bacterial cells are NOT treated with an antibiotic and NOT stained with fluorescent probes. In the negative control the bacterial cells are exposed to a killing agent AND stained with fluorescent probes. This is key to control the fluorescent compound quality to ascertain that when bacteria are damaged or even dead, the fluorescent agent will mark them.

Similarly, this is the first report of the innovative layouts of the panels designed by the inventors. However, it will be appreciated that the panel layouts reported can be changed within the scope of the present invention due to several reasons, such as the introduction of new drugs, a change in CLSI and EUCAST breakpoints, the introduction of different fluorochromes, or even a change of the sequence of drugs.

The outstanding results concerning the CA, EA and the few VME and ME reported in Table 9 are in conformity with the FDA and the ISO 20776-2 recommendations, demonstrating that the methods, assays and kits described herein can revolutionise antimicrobial therapeutic approaches worldwide, with significant clinical and public health impacts.

As shown in the multi-sites study, the FASTinov® kits consistently provide ultra-rapid AST (less than 2 hours) with high accuracy and reproducibility, resulting in an improved and timely diagnosis on septic patients, thereby impacting their clinical outcome.

### REFERENCES

1. WO2012/164547 A1. Kit and method of detecting the resistant microorganism to a therapeutic agent.
2. Puttaswamy S, et al 2011. Novel Electrical Method for Early Detection of Viable Bacteria in Blood Cultures, Journal of Clinical Microbiology Volume 49, Issue 6, June 2011, Pages 2286-2289.3.
3. Clinical and Laboratory Standards Institute. 2021. Performance Standards for Antimicrobial Susceptibility Testing: Twenty-eight Informational Supplement M100.
4. A novel flow cytometric assay for rapid detection of extended-spectrum beta-lactamases.aria-Ramos I, Espinar M J, Rocha R, Santos-Antunes J, Rodrigues A G, Cantón R, Pina-Vaz C. Clin Microbiol Infect. 2013 January; 19(1):E8-E15
5. Evaluation of FASTinov Ultrarapid Flow Cytometry Antimicrobial Susceptibility Testing Directly from Positive Blood Cultures. Silva-Dias A, Pérez-Viso B, Martins-Oliveira I, Gomes R, Rodrigues A G, Cantón R, Pina-Vaz C. J Clin Microbiol. 2021 Sep. 20; 59(10):e0054421. doi: 10.1128/JCM.00544-21. Epub 2021.
6. A Rapid Flow Cytometric Antimicrobial Susceptibility Assay (FASTvet) for Veterinary Use—Preliminary Data. Andrade F F, Gomes R, Martins-Oliveira I, Dias A, Rodrigues A G, Pina-Vaz C. Front Microbiol. 2020 Aug. 7; 11:1944. doi: 10.3389/fmicb.2020.01944. eCollection 2020.
7. Ultra-rapid flow cytometry assay for colistin MIC determination in Enterobacterales, Pseudomonas aeruginosa and Acinetobacter baumannii. Fonseca E Silva D, Andrade F F, Gomes R, Silva-Dias A, Martins-Oliveira I, Pérez-Viso B, Ramos M H, Rodrigues A G, Cantón R, Pina-Vaz C. Clin Microbiol Infect. 2020 November; 26(11):1559.e1-1559.e4. doi: 10.1016/j.cmi.2020.08.019. Epub 2020.
8. ISO, Standardization tIOf. In vitro diagnostic medical devices—Clinical performance studies using specimens from human subjects—Good study practice. ISO 20916:2019. 1 ed: ISO (the International Organization for Standardization); 2019.
9. ISO, Standardization tIOf. ISO 20776:2006 Clinical laboratory testing and in vitro diagnostic test systems: Susceptibility testing of infectious agents and evaluation of performance of antimicrobial susceptibility test devices.
10. European Committee on Antimicrobial Susceptibility Testing. EUCAST: Clinical breakpoints and dosing of antibiotics 2021.
11. M07: Dilution AST for Aerobically Grown Bacteria—CLSI 2021.
12. Testing ECoAS. EUCAST; guidelines for detection of resistance mechanisms and specific resistances of clinical and/or epidemiological importance. 2.0 ed2017.
13. Antifungal activity of local anesthetics against Candida species. C Pina-Vaz¹, A G Rodrigues, F Sansonetty, J Martinez-De-Oliveira, A F Fonseca, P A Mårdh, Infect Dis Obstet Gynecol.
14. Benzydamine Bactericidal Effect Results from a Permanent Cell Membrane Lesion. Ferdinando F Andrade^{1,2}, Irene Pina-Vaz³⁻⁴, Acácio G Rodrigues^{1,4}, Cidalla Pina-Vaz^{1,2}, Infect Dis Diag Treat 5: 169.
15 Roos V. Ulett G C, Schembri M A and Klemm P. The asymptomatic bacteriuria Escherichia coli strain 83972 outcompetes uropathogenic E. coli stains in human urine. Infect Immun 74 (1), 615-624.

1. A method for determining the susceptibility phenotype, to at least one therapeutic agent, of a microorganism present in a biological sample, the method comprising:
(i) introducing a biological sample comprising a microorganism into each of:
(a) one or more of a first, test reservoir,
(b) one or more of a second, positive control reservoir, and
(c) one or more of a third, negative control reservoir, wherein the negative control reservoir comprises non-viable microorganisms;
(ii) contacting the biological sample in the one or more first, test reservoir with at least one therapeutic agent;
(iii) contacting the biological sample in the one or more first, second and third reservoir with at least one fluorescent marker; and
(iv) performing a fluorescence analysis in order to obtain one or more fluorescence parameters for the biological sample in each of the reservoirs,
wherein the microorganism's susceptibility phenotype to the at least one therapeutic agent is obtained by comparing one or more fluorescence parameters between the reservoirs.

2. The method according to claim 1, wherein the one or more first test reservoir comprises viable or living microorganisms, optionally at least 60%, 70%, or 80% viable cells.

3. The method according to either claim 1 or 2, wherein the one or more second positive control reservoir comprises viable or living microorganisms, optionally at least 60%, 70%, or 80% viable cells.

4. The method according to any preceding claim, wherein the one or more third negative control reservoir are rendered non-viable by exposure to a cell-killing agent, optionally wherein the one or more third negative control reservoir comprises the cell-killing agent, optionally wherein the one or more third negative control reservoir comprises at least 60%, 70%, or 80% non-viable cells.

5. The method according to claim 4, wherein the cell-killing agent is selected from a group consisting of: ethanol, 2-phenoxyethanol, citric acid, and benzydamine hydrochloride.

6. The method according to claim 5, wherein the cell-killing agent is benzydamine hydrochloride.

7. The method according to any preceding claim, wherein the susceptibility phenotype is susceptible, intermediate or resistant.

8. The method according to any preceding claim, wherein the method further comprises introducing the biological sample in one or more of a fourth auto-fluorescence control reservoir, which does not comprise the therapeutic agent or the fluorescent marker.

9. The method according to claim 8, wherein the one or more fourth auto-fluorescence control reservoir comprises viable or living microorganisms, optionally at least 60%, 70%, or 80% viable cells.

10. The method according to any preceding claim, wherein the method further comprises a sample preparation step.

11. The method according to claim 10, wherein the sample preparation step comprises purification of the microorganism from the biological sample before it is introduced into each of the first, second, third and/or optionally the fourth reservoir.

12. The method according to either claim 10 or 11, wherein the sample preparation step comprises:
(i) obtaining the biological sample comprising the microorganism; and
(ii) contacting the sample with a density gradient solution.

13. The method according to claim 12, wherein the density gradient solution is Histopaque®.

14. The method according to claim 12 or 13, wherein the sample preparation step comprises contacting the biological sample with a haemolytic agent before it is contacted with the density gradient solution, optionally wherein the haemolytic agent is Triton X-100 or Tergitol.

15. The method according to any one of claims 10-14, wherein the sample preparation step further comprises:
(iii) identifying the microorganism before introducing the biological sample comprising the microorganism into each of the one or more first, second, third and/or optionally the fourth reservoir.

16. The method according to claim 15, wherein the identification of the microorganism is performed using a mass spectrometry method, preferably mass spectrometry MALDI-TOF, optionally wherein the biological sample is dried prior to the identification of the microorganism, optionally wherein the drying of the biological sample is achieved by a centrifugation step and/or an air-drying process.

17. The method according to any preceding claim, wherein the biological sample is of a human, animal or environmental origin, preferably wherein the sample is of a human origin.

18. The method according to claim 17, wherein the biological sample of human origin is a urine sample or blood sample, preferably a blood sample.

19. The method according to any preceding claim, wherein the microorganism is selected from the group consisting of: a bacterium, a virus, a fungus or a protozoan, preferably wherein the microorganism is a bacterium.

20. The method according to any preceding claim, wherein the therapeutic agent is selected from the group consisting of: an antibiotic, an antiviral, an antifungal; and an antiprotozoan agent, preferably wherein the therapeutic agent is an antibiotic.

21. The method according to any preceding claim, wherein the fluorescent analysis is a flow cytometry analysis or a laser scanning analysis, preferably wherein the fluorescent analysis is a flow cytometry analysis.

22. The method according to any preceding claim, wherein the one or more flow cytometric parameters comprise forward scatter and/or side scatter and/or fluorescence parameters.

23. The method according to claim 22, wherein the fluorescence scatter signal is intensity, spectral profile and/or cell count.

24. The method according to any preceding claim, wherein the fluorescent marker is selected from a group consisting of: acridine dye; cyanine dye; fluorone dye; oxazin dye; phenanthridine dye; and a rhodamine dye.

25. The method according to any preceding claim, wherein the fluorescent marker is selected from the group consisting of: CTC (5-Cyano-2,3-ditolyl tetrazolium chloride), Calcein AM, Dihydrorhodamine 123, DIBAC4(3), DioC 2(3), Fluorescein Diacetate, 5CFDA, AM, CFDA-SE, Propidium Iodine, SYTO 16 Green Fluorescent, and Nucleic Acid Stain.

26. The method according to any preceding claim, wherein the reservoir is a container, a tube or a well, preferably wherein the reservoir is a well.

27. A kit for use in the method of any one of claims 1-26.

28. The kit according to claim 27, wherein the kit is a panel comprising the one or more first, second, the third, and/or the fourth reservoirs, optionally wherein the panel is a 96-well plate.

29. The kit according to either claim 27 or 28, wherein the panel is configured to analyse gram-negative bacteria.

30. The kit according to claim 29, wherein the panel is the panel substantially as shown in FIG. 3A or FIG. 5A.

31. The kit according to claim 27 or 28, wherein the panel is configured to analyse gram-positive bacteria.

32. The kit according to claim 31, wherein the panel is the panel substantially as shown in FIG. 4A.

33. A method of purifying a microorganism from a biological sample comprising:
(i) obtaining a biological sample comprising a microorganism; and
(ii) contacting the sample with a density gradient solution, to thereby purify the microorganism.

34. The method according to claim 33, wherein the method further comprises contacting the biological sample with a haemolytic agent prior to contacting the sample with the density gradient solution.

35. The method according to claim 33, wherein the biological sample is a blood sample.

36. The method according to either claim 34 or 35, wherein the haemolytic agent is Triton X-100 or Tergitol.

37. The method according to any one of claims 33-36, wherein the density gradient solution is Histopaque®.

38. Use of a density gradient solution, for purifying a microorganism from a biological sample.

39. The use according to claim 38, wherein the density gradient solution is Histopaque®.
